(19) United States
(12) Patent Application Publication
(10) Pub. No.: US 2026/0242638 A1
(43) Pub. Date: Aug. 20, 2026

(54) HEXAGONAL SELF-ADHESIVE LAYER STRUCTURE

(71) Applicant: LTS LOHMANN THERAPIE-SYSTEME AG, Andernach (DE)

(72) Inventor: Stefan LORSCHEIDT, Bonn (DE)

(21) Appl. No.: 19/164,735

(22) PCT Filed: Mar. 15, 2024
(86) PCT No.: PCT/EP2024/056988

§ 371 Date: Sep. 12, 2025

(30) Foreign Application Priority Data
Mar. 15, 2023 (EP) 23162162.4

(51) Int. Cl. C09J 7/29; A61K 9/70; A61K 31/165; C09J 183/04

(52) U.S. Cl. CPC C09J 7/29; A61K 9/7084; A61K 31/165; C09J 183/04

(57) ABSTRACT

The present invention relates to a self-adhesive layer structure for use in a medical patch, having a hexagonal shape that comprises at least one hexagon, a medical patch and a sheet of medical patches comprising such self-adhesive layer structure(s) as well as such medical patches for use in a method of treatment and processes of manufacture of such medical patches.

### TECHNICAL FIELD OF THE INVENTION

The present invention relates to a self-adhesive layer structure for use in a medical patch, having a hexagonal shape which comprises at least one hexagon. Further, the present invention relates to a medical patch comprising the self-adhesive layer structure and to a sheet of medical patches comprising two or more of the self-adhesive layer structures.

### BACKGROUND OF THE INVENTION

Medical patches are adhesive patches placed on the skin of a patient to deliver a specific dose of medication through the skin.

Medications have been topically applied to the skin for thousands of years to treat local conditions. More recently, transdermal delivery technology has been developed to treat a range of conditions beyond the local site of application. While topical delivery of a compound and/or drug involves only minimal penetration of the skin layer and thus avoids systemic effects, transdermal medications refer to pharmaceutical compounds that are applied on the skin, but cross the outermost layer of it (the skin barrier) to get into the blood stream and/or for targeting an effect on more distant tissues or organs.

Accordingly, the body location for the medical patch application may vary with the therapeutic category of the drug contained therein. Sites to place transdermal medical patches as being systemically effective usually include large and even surfaces, such as upper arm, chest or back of the patient. In contrast, topical medical patches have to be applied at the site of the condition to be treated, for example at the hands or feet of the patient. Such application sites are particularly challenging due to their uneven surface and complexity, and need to be considered in view of shape and size of the medical patch.

However, medical patches and in particular topical medical patches are commonly only available as large-scale patches which need to be trimmed based on the application site and/or can hardly be applied without wrinkling in order to provide a seamless full coverage of the application site. This is time-consuming and requires increased adhering skills of the applying person. Wrinkling not only affects wearing comfort and aesthetics of the application site but the loss of contact area potentially results in less active being utilized, and also in reduced adhesion of the patch and thus is undesirable. In addition, trimming or cutting the patch bears the risk of contamination as the active agent-containing adhesive layer potentially comes in contact with the cutting tool and/or the hands of the person handling the patch.

For example, the 8% capsaicin patch dermal delivery system administered under the brand name. QUTENZA® (Grünenthal) is suggested to be cut to match the size and shape of the treatment area. QUTENZA® originally covers a rectangular area of 14 cm×20 cm. The topical system is indicated in adults for the treatment of neuropathic pain associated with postherpetic neuralgia (PHN) as well as for neuropathic pain associated with diabetic peripheral neuropathy (DPN) of the feet, and is currently approved for use in adults in the treatment of post-surgical neuropathic pain (PSNP). Thus, it is frequently used to be applied at hands and fingers or lower legs and feet, and thus has to be trimmed to small and uneven application surfaces.

It is therefore desirable to provide a self-adhesive layer structure for a medical patch, which provides for simplified coverage of skin areas to be treated, in particular of challenging and uneven surfaces.

### OBJECTS AND SUMMARY OF THE INVENTION

It is an object of the present invention to provide a self-adhesive layer structure for a medical patch, which is improved in comparison to the patches described in the prior art.

It is a further object of the present invention to provide a self-adhesive layer structure for a medical patch, which provides easy and time-efficient handling.

It is a further object of the present invention to provide a self-adhesive layer structure for a medical patch, which allows for simplified coverage of application sites. In particular, the object is to provide a self-adhesive layer structure for a medical patch, enabling simplified coverage of uneven applications sites without the need of cutting the medical patch before application. In particular, the object is to provide a self-adhesive layer structure for a medical patch which is improved in terms of facilitating a seamless and full coverage of application sites.

It is a further object of the present invention to provide a self-adhesive layer structure for a medical patch with reduced wrinkling during application. In particular, the object is to provide a self-adhesive layer structure for a medical patch, providing full adhesiveness even at complicated skin areas.

It is a further object of the present invention to provide a self-adhesive layer structure for a medical patch, which simplifies surrounding of fingers or toes.

It is also an object of the present invention to provide a self-adhesive layer structure for a medical patch, which allows for covering of larger skin areas without leaving gaps.

These objects and others are accomplished by the present invention, which according to one aspect relates to a self-adhesive layer structure for use in a medical patch, having a hexagonal shape and comprising:
A) a backing layer; and
B) an active layer comprising a polymer I and an active agent;
wherein
   the backing layer and the active layer are coextensive and provide the hexagonal shape of the self-adhesive layer structure, and
   the hexagonal shape comprises at least one hexagon, wherein
      all pairs of opposite sides of the hexagon are parallel, and
      the sides of the hexagon have a length of from 0.2 to 10 cm.

It has been surprisingly found that the self-adhesive layer structure according to the present invention, which has a hexagonal shape comprising at least one hexagon with a length of from 0.2 to 10 cm, has advantageous properties regarding improved coverage of small and/or uneven application sites of the human body. In particular, it has been found that the hexagonal shape allows for simplified adhering and reducing wrinkling without the need of cutting the medical patch before application. Thus, the medical patch is even suitable for problematic applications sites, such as, e.g., hands or feet.

According to certain embodiments of the invention, the present invention relates to a medical patch comprising
the self-adhesive layer structure as described herein and
a release liner,
wherein the release liner is coextensive with the self-adhesive layer structure or extends beyond the boundary of the self-adhesive layer structure in all directions.

According to certain embodiments of the invention, the present invention relates to a sheet of medical patches comprising
two or more self-adhesive layer structures as described herein and
a release liner,
wherein the release liner is coextensive with the self-adhesive layer structures or extends beyond the boundary formed by all self-adhesive layer structures in all directions.

### Definitions

Within the meaning of this invention, the term “medical patch” refers to a dermal delivery system by which the active agent is administered to a patient, and which comprises an effective amount of the active agent in a self-adhesive layer structure located on a detachable protective layer (release liner). In this context, the term “medical patch” is understood to mean an adhesive patch which can be a topical medical patch or a transdermal therapeutic system (TTS). Even if the topical medical patch as well as the TTS are topically applied in the sense that they are attached to the skin of the patient, the term “topical” or “topical administration” refers to the administration of the active agent relying on passive diffusion into the skin itself, which creates a local effect at a point of action. In contrast, the term “TTS” refers to a system by which the active agent is administered to the systemic circulation via transdermal delivery.

Within the meaning of this invention, the term “self-adhesive layer structure” refers to the active agent-containing structure providing the area of release for the active agent during administration. It is “self-adhesive” and thus provides adhesion to the skin so that typically no further aid for fixation on the skin is needed. The self-adhesive layer structure comprises a backing layer and an active layer, as well as optionally a skin contact layer as described herein. The self-adhesive layer structure thus comprises an effective amount of the active agent.

As used herein, the expression “active agent” refers to any substance of interest to be delivered by the self-adhesive layer structure to provide a beneficial or desirable effect on the condition of the subject's body either systemically or locally at the delivery site. An active agent in particular includes biologically or pharmacologically active compounds, which may also be referred to as active, drug substance, drug, active ingredient, active pharmaceutical ingredient (API), or the like. In this context, the term “effective amount” or “therapeutically effective amount” refers to a quantity of active agent in the self-adhesive layer structure sufficient to provide, if administered by the medical patch to a patient, the desired (therapeutic) effect such as pain relief/reduction. A TTS usually contains more active agent in the system than is in fact provided to the skin and the systemic circulation, which is usually necessary to provide enough driving force for the delivery from the TTS to the systemic circulation.

Within the meaning of this invention, the terms “active”, “active agent” and the like refer to the respective active agent in any pharmaceutically acceptable chemical and morphological form and physical state. These forms include without limitation the active agent in its free base/free acid form, protonated or partially protonated form, deprotonated or partially deprotonated form, salts, cocrystals and in particular acid/base addition salts formed by addition of an inorganic or organic acid/base such as hydrochloride or tartrate salts, solvates, hydrates, clathrates, complexes and so on, as well as the active agent in the form of particles, which may be micronized, crystalline and/or amorphous, and any mixtures of the aforementioned forms.

The active agent, where contained in a medium such as a solvent, may be dissolved or dispersed or in part dissolved and in part dispersed.

When the active agent is mentioned to be used in a particular form in the manufacture of the medical patch, this does not exclude interactions between this form of the active agent and other ingredients of the self-adhesive layer structure, e.g. salt formation or complexation, in the final medical patch. This means that, even if the active agent is included in its free base/acid form, it may be present in the final medical patch in protonated or partially protonated/or deprotonated or partially deprotonated form or in the form of an acid addition salt, or, if it is included in the form of a salt, parts of it may be present as free base in the final medical patch. Unless otherwise indicated, in particular the amount of the active agent in the self-adhesive layer structure relates to the amount of the active agent included in the medical patch during manufacture of the medical patch and is calculated based on the active agent itself, but not on other forms thereof. The active agent starting material included in the medical patch during manufacture of the medical patch may be in the form of particles. The active agent may e.g. be present in the self-adhesive layer structure in the form of particles and/or dissolved.

In this context, the term “particles” refers to a solid, particulate material comprising individual particles, the dimensions of which are negligible compared to the material. In particular, the particles are solid, including plastic/deformable solids, including amorphous and crystalline materials. The term “dispersing” refers to a step or a combination of steps wherein a starting material (e.g. the active agent) is not totally dissolved. Dispersing in the sense of the invention comprises the dissolution of a part of the starting material (e.g. active agent particles), depending on the solubility of the starting material (e.g. the solubility of the active agent in the coating composition).

There are two main types of medical patches using (passive) active agent delivery, i.e. matrix-type medical patches and reservoir-type medical patches. The release of the active agent in a matrix-type medical patch is mainly controlled by the matrix including the active agent itself. In contrast thereto, a reservoir-type medical patch typically needs a rate-controlling membrane controlling the release of the active agent. In principle, also a matrix-type medical patch may contain a rate-controlling membrane. However, matrix-type medical patches are advantageous in that, compared to reservoir-type medical patches, usually no rate determining membranes are necessary and no dose dumping can occur due to membrane rupture. In summary, matrix-type medical patches are less complex in manufacture and easy and convenient to be used.

In this context, a “matrix-type medical patch” is understood to mean a system or structure wherein the active agent is homogeneously dissolved and/or dispersed within a polymeric carrier, i.e. the matrix, which forms with the active agent and optionally remaining ingredients a matrix layer. In such a system, the matrix layer controls the release of the active agent from the medical patch. Preferably, the matrix layer has sufficient cohesion to be self-supporting so that no sealing between other layers is required. Accordingly, the active layer may be an active matrix layer, wherein the active agent is homogeneously distributed within a polymer matrix. The active matrix layer may comprise two active agent-containing matrix layers, which may be laminated together. Matrix-type medical patches may in particular be in the form of a “drug-in-adhesive”-type medical patch referring to a system wherein the active agent is homogeneously dissolved and/or dispersed within a pressure sensitive adhesive matrix. In this connection, the active matrix layer may also be an active pressure sensitive adhesive layer or active pressure sensitive adhesive matrix layer. A medical patch comprising the active agent dissolved and/or dispersed within a polymeric gel, e.g. a hydrogel, is also considered to be of matrix-type in accordance with present invention.

Medical patches with a liquid active agent-containing reservoir are referred to by the term “reservoir-type medical patch”. In such a system, the release of the active agent is preferably controlled by a rate-controlling membrane. In particular, the reservoir is sealed between the backing layer and the rate-controlling membrane. Accordingly, the active layer may be an active reservoir layer, which preferably comprises a liquid reservoir comprising the active agent, and wherein the active reservoir layer and the skin contact layer may be separated by the rate-controlling membrane. In the active reservoir layer, the active agent is preferably dissolved in a solvent such as ethanol or water or in silicone oil.

Reservoir-type medical patches are not to be understood as being of matrix-type within the meaning of the invention. However, microreservoir-type medical patches (biphasic systems having deposits (e.g. spheres, droplets) of an inner active agent-containing phase dispersed in an outer polymer phase), considered in the art to be a mixed form of a matrix-type medical patch and a reservoir-type medical patch that differ from a homogeneous single phase matrix-type medical patch and a reservoir-type medical patch in the concept of drug transport and drug delivery, are considered to be of matrix-type within the meaning of the present invention.

Thus, a microreservoir-type medical patch refers to a microreservoir systems, in which a liquid active agent preparation is dispersed in an adhesive matrix in the form of small droplets (“microreservoirs”). The size of the resulting droplets depends on the stirring conditions and the applied shear forces during stirring. It can be determined by an optical microscopic measurement (for example by Leica MZ16 including a camera, for example Leica DSC320) by taking pictures of the microreservoirs at different positions at an enhancement factor between 10 and 400 times, depending on the required limit of detection. By using imaging analysis software, the sizes of the microreservoirs can be determined. Microreservoirs systems are disclosed in U.S. Pat. Nos. 3,946,106, 4,053,580, 4,814,184 and 5,145,682, each of which is incorporated herein by reference. Specific microreservoirs systems are described in international patent publication WO0101967 the disclosure of which is incorporated herein by reference. These microreservoir systems contain, as base polymer, polysiloxanes and amphiphilic solvents for the microreservoir droplets.

The self-adhesive layer structure may be a pressure sensitive adhesive layer structure.

Within the meaning of this invention, the term “pressure-sensitive adhesive” (also abbreviated as “PSA”) refers to a material that in particular adheres with finger pressure, is permanently tacky, exerts a strong holding force and should be removable from smooth surfaces without leaving a residue. It is obtainable from a solvent-containing adhesive coating composition after coating on a film and evaporating the solvents (e.g. n-heptane or ethyl acetate). In this context, the term “solvent” is understood to mean any liquid substance, which preferably is a volatile organic liquid such as methanol, ethanol, isopropanol, acetone, ethyl acetate, methylene chloride, hexane, n-heptane, toluene and mixtures thereof. A pressure sensitive adhesive layer, when in contact with the skin, is self-adhesive. According to certain embodiments, the self-adhesive layer structure according to the invention includes a pressure sensitive adhesive layer for skin contact which may be provided in the form of a pressure sensitive adhesive matrix or in the form of an additional layer, i.e. a pressure sensitive adhesive skin contact layer. An adhesive overlay may still be employed to advance adhesion.

Within the meaning of this invention, the term “active layer” refers to a layer containing the active agent (active agent-containing layer) and providing the area of release. The term covers active agent-containing reservoir layers (active reservoir layer) and active agent-containing matrix layers (active matrix layer), and in particular active agent-containing microreservoir layers (active microreservoir layers). If the active layer is an active matrix layer, said layer is present in a matrix-type medical patch. As used herein, the active layer is preferably an active matrix layer, and it is referred to the final solidified layer, e.g. obtained after coating and drying a solvent-containing coating composition as described herein. Alternatively, an active matrix layer is obtained after melt-coating and cooling. The active matrix layer may also be manufactured by laminating two or more such solidified layers (e.g. dried or cooled layers) of the same composition to provide the desired area weight. According to certain embodiments, the matrix layer is a pressure sensitive adhesive matrix layer.

Within the meaning of this invention, the term “skin contact layer” refers to a layer that may be included in the self-adhesive layer structure to be in direct contact with the skin of the patient during administration. In this case, the other layers of the self-adhesive layer structure do not contact the skin and do not necessarily have self-adhesive properties. The skin contact layer is directly attached to the active layer, or a membrane is located between the active layer and the skin contact layer. In this context, the term “membrane” is understood to mean a layer, which is provided between the active layer and the skin contact layer and is at least semipermeable for the active agent. The membrane may be a microporous film or a nonporous partition membrane. Preferred membranes can be selected from the group consisting of polyethylene membranes, polyurethane coated polyethylene terephthalate/polyethylene membranes, polyurethane membranes, and ethylene vinyl acetate membranes. The additional skin contact layer is preferably present as adhesive layer.

Within the meaning of the invention, the term “backing layer” refers to a layer which supports the active layer. At least one backing layer in the medical patch self-adhesive layer structure and usually the backing layer of the active layer is substantially impermeable to the active agent, as well as optionally any additive, contained in the layer during the period of storage and administration and thus prevents active loss or cross-contamination in accordance with regulatory requirements. According to certain embodiments, the backing layer is also occlusive, meaning substantially impermeable to water and water-vapor. Suitable materials for a backing layer include polyethylene terephthalate (PET), polyethylene (PE), ethylene vinyl acetate-copolymer (EVA), polyesters, polyurethanes, and mixtures thereof. Suitable backing layers may be siliconized in order to improve the adhesion of the active layer to the backing layer.

Furthermore, an adhesive overlay may be present. In this context, the term “adhesive overlay” is understood to mean a self-adhesive layer structure that is free of the active agent and larger in area than the self-adhesive layer structure and provides additional area adhering to the skin, but no area of release of the active agent. It enhances thereby the overall adhesive properties of the self-adhesive layer structure or the medical patch. The area of said adhesive overlay adds to the overall size of the medical patch but does not add to the area of release. The adhesive overlay may comprise a self-adhesive polymer or a self-adhesive polymer mixture selected from the group of acrylic polymers, polyisobutylenes, styrene-isoprene-styrene copolymers, polysiloxanes, and mixtures thereof, which may be identical to or different from any polymer or polymer mixture included in the self-adhesive layer structure. The adhesive overlay comprises a backing layer that may provide occlusive or non-occlusive properties and an adhesive layer. According to certain embodiments, the backing layer of the adhesive overlay provides non-occlusive properties.

Within the meaning of this invention, the term “area weight” refers to the dry weight of a specific layer, e.g. of the active layer, provided in g/m². The area weight values are subject to a tolerance of ±10%, ±7.5%, or ±5% due to manufacturing variability.

If not indicated otherwise “%” refers to wt. % (% by weight).

Within the meaning of this invention, the term “polymer”, e.g., polymer I or II, refers to any substance consisting of so-called repeating units obtained by polymerizing one or more monomers, and includes homopolymers which consist of one type of monomer and copolymers which consist of two or more types of monomers. Polymers may be of any architecture such as linear polymers, star polymer, comb polymers, brush polymers, of any monomer arrangements in case of copolymers, e.g. alternating, statistical, block copolymers, or graft polymers. The minimum molecular weight varies depending on the polymer type and is known to the skilled person. Polymers may e.g. have a molecular weight above 2000, above 5000, or above 10,000 Dalton. Correspondingly, compounds with a molecular weight below 2000, below 5000, or below 10,000 Dalton are usually referred to as oligomers.

Within the meaning of the invention, the term “silicone-based polymer” refers to a non-hybrid polymer (i.e. a polymer, which does not include a hybrid species) comprising polysiloxanes. Polysiloxanes can be made from solvent-free two-component systems or a solution in organic solvents. They exist in two fundamentally different variants: polysiloxanes which have free silanol groups and amine resistant polysiloxanes which are distinguished in that the free silanol groups are derivatized by trimethylsilyl groups. The methyl groups can be completely or partially replaced by other alkyl radicals or alternatively phenyl radicals. Polysiloxanes as used herein are synthesized from linear bifunctional and branched polyfunctional oligomers, the ratio of which determines the physical properties thereof. More polyfunctional oligomers result in a more cross-linked adhesive with a higher cohesion and a reduced tack, less polyfunctional oligomers result in a higher tack and a reduced cohesion. It is preferred for the silicone-based polymer to be a mixture of high tack and medium tack, or high tack and low tack, polysiloxanes. According to certain embodiments, the at least one silicone-based polymer is a silicone-based pressure sensitive adhesive.

Within the meaning of the invention, the term “acrylic polymer” refers to a non-hybrid polymer based on acrylates. It may be a polymer obtainable from one or more monomers selected from acrylic acid, butylacrylate, 2-ethylhexylacrylate, glycidylmethacrylate, 2-hydroxyethyl-acrylate, methylacrylate, methylmethacrylate, butylmethacrylate, t-octylacrylamide, and vinylacetate.

Within the meaning of the invention, the term “silicone-acrylic hybrid polymers” refers to a hybrid polymer based on silicones and acrylates in the form of a pressure-sensitive adhesive. Silicone acrylic hybrid pressure-sensitive adhesives are described, for example, in EP 2 599 847 and WO 2016/130408. It was found that, depending on the solvent in which the silicone acrylic hybrid PSA is supplied, the arrangement of the silicone phase and the acrylic phase providing a silicone or acrylic continuous external phase and a corresponding discontinuous internal phase is different. If the silicone acrylic hybrid PSA is supplied in n-heptane, the composition contains a continuous, silicone external phase and a discontinuous, acrylic internal phase. If the silicone acrylic hybrid PSA composition is supplied in ethyl acetate, the composition contains a continuous, acrylic external phase and a discontinuous, silicone internal phase.

Within the meaning of the invention, the term “natural or synthetic rubbers” refers to an elastomer which is obtainable by polymerizing an unsaturated hydrocarbon, such as isoprene (2-methyl-1,3-butadiene), or by copolymerizing such hydrocarbons with styrene, butadiene, or the like. It includes natural and synthetic polyisoprene, polybutylene and polyisobutylene, styrene/butadiene polymers, styrene-isoprene-styrene block copolymers, hydrocarbon polymers such as butyl rubber, halogen-containing polymers such as polyacrylic-nitrile, polytetrafluoroethylene, polyvinylchloride, polyvinylidene chloride, and polychlorodiene, as well as other copolymers thereof. In certain embodiments, natural or synthetic rubbers may be styrenic triblock copolymers or polyisobutylenes.

Within the meaning of the invention, the term “polyisobutylenes” refers to polymers obtained by polymerization of isobutene.

Within the meaning of the invention, the term “styrene-isoprene-styrene block copolymers” refers to polymers obtained by living ionic copolymerization by sequentially introducing styrene, 2-methyl-1,3-butadiene (isoprene), and styrene into the reactor. The styrene content typically varies between 15 and 40%.

Within the meaning of this invention, the term “silicone gel adhesive” refers to an elastic, jelly-like material formed by lightly crosslinking silicone polymers. It may be prepared from a gel producing composition as described further below upon curing. In particular, the silicone gel adhesive forms upon curing of polysiloxanes comprising reactive groups such as Si—H reactive groups and aliphatic unsaturated groups, which react with each other in the presence of a hydrosilylation catalyst. According to certain embodiments, the silicone gel adhesive is based on a polydimethylsiloxane network, which may be formed in an addition reaction (hydrosilylation) between vinyl function polydimethylsiloxane groups (polymer) and hydrogen functional siloxanes (cross-linker). Accordingly, the silicone gel adhesive is typically applied by using a curable gel producing (2-component) composition, which solidifies upon curing.

Within the meaning of this invention, the term “saturation concentration” refers to that active agent concentration corresponding to the equilibrium state in which the solvent (i.e. the polymer II of the skin contact layer) cannot dissolve further solute (i.e. the active agent), and as a result the solid solute is present in equilibrium with the solid solution at defined temperature (room temperature—unmodified temperature found indoors in the laboratory where experiments are conducted and usually lies within 15 to 35° C., or about 18 to 25° C.). The saturation concentration of the active agent can be indicated in % by weight, based on the total weight of the active agent layer or skin contact layer, respectively. The saturation concentration can be determined e.g. using a method described by Liu, P., Gargiulo, P., Wong, J., and Novartis. Pharm. Research. Vol. 14, p. 317 (1997), herein referred to as “Sandwich method”, in which a multi-layered laminate is prepared comprising an upper and lower protective layer sandwiching a donor layer and an acceptor layer separated by a partitioning membrane that is permeable to the active agent. Since the donor layer contains an excess of the active agent and the acceptor layer is substantially free of the active agent, the active agent diffuses out of the donor layer through the partitioning membrane into the acceptor layer until the saturation concentration is achieved. The donor layer and the acceptor layer are manufactured from the respective polymer II of the skin contact layer. The donor layer is oversaturated with the active agent, while the acceptor layer is prepared analogously to the donor layer but does not comprise the active agent. The sandwich systems prepared are stored for a certain time, e.g., 7 days, at room temperature, to allow the active agent to diffuse from the donor layer into the acceptor layer. Then, the remaining active agent concentration of the donor layer is determined by means of HPLC (high performance liquid chromatography) in order to finally obtain the saturation concentration of the active agent in the respective polymer II of the skin contact layer.

As used herein, solubility parameters (SPs) are defined as the sum of all the intermolecular attractive forces, which, as a numerical estimate, are empirically related to the extent of mutual solubility of chemical species. The most convenient method to determine solubility parameters is Hildebrand's method, which computes the solubility parameter from molecular weight, boiling point and density data, which are commonly available for many materials: SP=(ΔEᵥ/V)^{1/2}, where V=molecular weight/density and ΔEᵥ=energy of vaporization. For materials, such as high molecular weight polymers, which have vapor pressures too low to detect, several methods have been developed which use the summation of atomic and group contributions to vaporization. Such a method of calculating the solubility parameter of a material has been described e.g. by Small, J. Applied Chem. Vol. 3, p. 71 (1953). Some solubility parameters (calculated by Small's method) of exemplary polymers useful in the practice of the invention are as follows: Polydimethylsiloxane 14.9 MPa^{1/2}, polyisobutylene 15.7 MPa^{1/2}, polyethylene/butylene 16.2 MPa^{1/2}, polyisoprene 16.6 MPa^{1/2}, polyethylene 16.6 MPa^{1/2} polybutadiene 16.6 MPa^{1/2}, polybutadiene-co-styrene (75/25 to 72/28) 17.4 MPa^{1/2}, polystyrene 18.6 MPa^{1/2}, polymethyl methacrylate 19.0 MPa^{1/2}, polymethyl acrylate 19.8 MPa^{1/2}.

Within the meaning of the invention, the term “soluble polyvinylpyrrolidone” refers to polyvinylpyrrolidone, also known as povidone, which is soluble with more than 10% in at least ethanol, preferably also in water, diethylene glycol, methanol, n-propanol, 2 propanol, n-butanol, chloroform, methylene chloride, 2-pyrrolidone, macrogol 400, 1,2 propylene glycol, 1,4 butanediol, glycerol, triethanolamine, propionic acid and acetic acid. Examples of polyvinylpyrrolidones which are commercially available include Kollidon® 12 PF, Kollidon® 17 PF, Kollidon® 25, Kollidon® 30 and Kollidon® 90 F supplied by BASF, or povidone K90F. The different grades of Kollidon® are defined in terms of the K-Value reflecting the average molecular weight of the polyvinyl-pyrrolidone grades. Kollidon® 12 PF is characterized by a K-Value range of 10.2 to 13.8, corresponding to a nominal K-Value of 12. Kollidon® 17 PF is characterized by a K-Value range of 15.3 to 18.4, corresponding to a nominal K-Value of 17. Kollidon® 25 is characterized by a K-Value range of 22.5 to 27.0, corresponding to a nominal K-Value of 25, Kollidon® 30 is characterized by a K-Value range of 27.0 to 32.4, corresponding to a nominal K-Value of 30. Kollidon® 90 F is characterized by a K-Value range of 81.0 to 97.2, corresponding to a nominal K-Value of 90. Preferred Kollidon® grades are Kollidon® 12 PF, Kollidon® 30 and Kollidon® 90 F. In this contex, the term “K-Value” refers to a value calculated from the relative viscosity of polyvinylpyrrolidone in water according to the European Pharmacopoeia (Ph.Eur.) and USP monographs for “Povidone”. For all grades and types of polyvinylpyrrolidone, it is preferred that the amount of peroxides is within certain limits, in particular, the peroxide amount is equal to or less than 500 ppm, more preferably equal to or less than 150 ppm, and most preferably equal to or less than 100 ppm.

Within the meaning of the invention, the term “hexagonal shape” refers to the two-dimensional shape of the self-adhesive layer structure which is provided by the backing layer and the active layer, or the backing layer, the active layer and the skin contact layer, respectively, and which can be seen when regarding the self-adhesive layer structure from above onto the backing layer. A hexagonal shape in the sense of the present invention is understood as any shape that can be formed by one hexagon or by an aggregation of two or more hexagons. This means, the hexagonal shape according to the invention as a whole does not need to have the shape of a hexagon, but is to be constituted by at least one hexagon. The vertexes of the hexagonal shape may be sharp or rounded. If two or more hexagons constitute the hexagonal shape, they may be integrally connected to each other, i.e. only separable by e.g. cutting the self-adhesive layer structure, or may be detachably connected to each other using e.g. a perforated line. The backing layer and the active layer, or the backing layer, the active layer and the skin contact layer, respectively, as well as optionally the membrane, are coextensive, i.e. they have the same planar extent and/or share the same boundary. In other words, the backing layer and the active layer, or the backing layer, the active layer and the skin contact layer, respectively, as well as optionally the membrane, each provides a shape in the form of at least one congruent hexagon.

Within the meaning of the invention, the term “hexagon” refers to a six-sided polygon. In a “convex hexagon”, each of the six points where two sides of the hexagon meet in pairs (vertices) points outwards. Two adjacent vertices are respectively connected by one of the six sides (common side). Non-adjacent vertices can be connected by one of nine diagonals lying inside the closed hexagonal chain (boundary) of the convex hexagon. A convex hexagon requires the least total length of the boundary, compared to other polygons having the same area.

A convex hexagon may also be described as a six-sided polygon having interior angles (vertex angles) which are each less than 180°. The total of the interior angles of any simple (non-self-intersecting) hexagon is 720°. Thus, a (convex) hexagon whose vertex angles are equal to 120° each is also referred to as being equiangular. A (convex) hexagon having all sides of equal length is also referred to as being equilateral. If a (convex) hexagon is both equilateral and equiangular, it is also referred to as being regular.

The (convex) hexagon may be symmetrical, in particular mirror symmetrical or rotational symmetrical. In this context, mirror symmetry, also referred to as reflection symmetry, is understood to mean symmetry with respect to a reflection. Such symmetric function of a two-dimensional shape is that if the shape were to be folded half over the mirror axis, the two halves would be identical: the two halves are each other's mirror images. Thus, a regular hexagon has six axes of symmetry, because there are six different ways to fold it and have the sides all match. Rotational symmetry of order n, also called n-fold rotational symmetry, of a two-dimensional shape with respect to a particular point is understood to mean that rotation by an angle of 360°/n (180°, 120°, 90°, 72°, 60°, etc.) does not change the shape. Thus, a regular hexagon has rotational symmetry of order 6, because it looks the same after each of a partial turn about an angle of 60°.

Within the meaning of the invention, the term “parallelogon” refers to a (convex) hexagon wherein all pairs of opposite sides (two sides being separated from each other by the same number of sides in both boundary directions, i.e., by two sides in a hexagon) are parallel and the two sides of each pair of parallel opposite sides are equal in length. This includes a parallelogon having in total three different side length or having in total only two different side length, as well as a parallelogon having all sides of equal length. The term “parallelogon” includes, e.g., a shape obtained by elongating a parallelogram or a shape obtained by elongating a rhombus, and in particular by separating a parallelogram or rhombus, respectively, at two of its non-adjacent vertices and introducing a pair of parallel opposite sides of equal length. In this context, the term “parallelogram” is understood to mean a simple four-sided polygon with two pairs of parallel sides. If the four sides are of equal length, the parallelogram is also referred to as “rhombus”.

Within the meaning of the invention, the term “aspect ratio” refers to the height-to width ratio wherein the width and length are distances between two points on the boundary of the (convex) hexagon and the longer of the two distances is regarded as the width. In this context, the width of the (convex) hexagon is understood to mean the length of the longest distance between any two points on the boundary, which is often the length of the longest diagonal of the (convex) hexagon between two diametrically opposite vertices. The height of the (convex) hexagon is understood to mean the longest distance available between any two points on the boundary of the (convex) hexagon so that the line formed by connecting these two points is perpendicular to the line formed by connecting the two points defining the width (see above). The height-to-width-ratio corresponds to the ratio of inradius (radius of the inscribed circle) to circumradius (radius of the circumscribed circle), if available. The height-to width ratio of a regular hexagon is √3:2.

Regular hexagons fit together to tile the plane forming a honeycomb pattern. The honeycomb pattern is composed of regular hexagons arranged side by side, which tile the plan, i.e., completely fill the entire surface they span, so there are not any holes in between them. This is because the 120° angle is the angle at which the sides meet at the vertices when the hexagons are lined side by side, such that exactly three hexagons meeting at every vertex. The honeycomb pattern appears not only in honeycombs but also in many other places in nature, such as, e.g., in organic compounds (benzyl rings, proteins).

Hexagonal tiling, also referred to as hexagonal tessellation, is a regular tiling of the Euclidian plane, in which exactly three hexagons meet at each vertex. Besides using regular hexagons (FIG. 1a), hexagonal tiling may also be carried out with, for example, other (hexagonal) parallelogons, in particular with a parallelogon obtained by elongating a rhombus (FIG. 1b) or obtained by elongating a parallelogram (FIG. 1c). Such hexagonal shapes can tile the Euclidean plane by translation. Other hexagon shapes can tile the plane with different orientations. In this context, the term “tile the plane” is understood to mean the coverage of a particular plane (plane surface or curved surface) without leaving any gaps. Tiling the plane may be carried out with or without overlapping adjacent self-adhesive layer structures according to the present invention. Preferably, overlapping is avoided as much as possible.

Within the meaning of the invention, the term “sheet of medical patches” refers to a number of medical patches sharing a common release liner. Each of the medical patches represents an individual dosing unit that may be applied to the skin of the patient after peel-off from the release liner. The amount of active agent contained in the medical patch refers to the amount of active agent contained in the self-adhesive layer structure of the medical patch. The amount of active agent contained in the sheet of medical patches refers to the total amount of active agent contained in all self-adhesive layer structures of the medical patches constituting the sheet of medical patches. Accordingly, the area of release of the medical patch refers to the area provided by the self-adhesive layer structure of the medical patch, and the area of release of the sheet of medical patches refers to the area provided by all self-adhesive layer structures of the medical patches constituting the sheet of medical patches.

Within the meaning of the invention, the term “release liner” refers to a detachable protective layer, attached to the active layer or the skin contact layer of the self-adhesive layer structure(s). The release liner may have any suitable two-dimensional geometric shape and preferably has a polygonal shape, in particular a rectangular or square shape. According to certain embodiments, the area of the release liner encompasses the total area of all self-adhesive layer structures of the medical patches constituting the sheet of medical patches. In accordance with the invention, the release liner is coextensive with the self-adhesive layer structures or extends beyond the boundary formed by all self-adhesive layer structures in all directions, i.e. the polygonal chain formed by the outside sides of the hexagons of the self-adhesive layer structures completely lies within or on the polygonal chain formed by the sides of the release liner. Suitable release liners may be polyethylene terephthalate (PET) or polypropylene (PP) films, optionally provided with a silicone or fluoropolymer coating. This included, e.g., commercially available release liners, such as the Scotchpak® release liners 9741/9742/9744 of 3M.

Within the meaning of the invention, the term “weakened” refers to the result of any action (weakening) enabling easier separation of two sections of a self-adhesive layer structure or of two different self-adhesive layer structures, although the two sections/self-adhesive layer structures are still connected to each other. Such weakening may include but is not limited to folding, scratching, perforating, piercing, puncturing, punching, or cutting. According to certain embodiments, the weakening is carried out by perforation. In this context, the term “perforated” is understood to mean having small holes made in it. Perforation may be obtained by, e.g., needling or laser cutting.

Within the meaning of the invention, the term “fastening bridge” refers to a single point between two or three self-adhesive layer structures, in particular between two or three hexagonal shapes, such as two or three convex hexagons or two or three convex double hexagons, at which they are still connected, while the main part of the common sides is cut or weakened. It is preferably obtained by leaving the connection during the separation process, which may be carried out by, e.g. punching or cutting. The fastening bridge(s) enable(s) joint peel-off of self-adhesive layer structures connected in this manner from the release liner. In addition, the fastening bridge(s) is/are preferably so thin that they can easily be undone, e.g. by pulling on a part of the self-adhesive layer structures, in order to separate some of the self-adhesive layer structures from the others.

Within the meaning of the invention, the term “patient” refers to a subject who has presented a clinical manifestation of a particular symptom or symptoms suggesting the need for treatment, who is treated preventatively or prophylactically for a condition, or who has been diagnosed with a condition to be treated. Preferably, the patient suffers from neuropathic pain or mixed neuropathic and/or nociceptive pain such as joint pain or cancer pain.

The term “neuropathic pain” refers to pain caused by a lesion or disease of the somatosensory nervous system. In this context, the term “chronic neuropathic pain” is understood to mean neuropathic pain lasting for at least three months. When suffering from neuropathic pain, most patients complain of an ongoing or intermittent spontaneous pain of, e.g., burning, pricking, squeezing quality, which may be accompanied by evoked pain, particular to light touch and cold. Ectopic activity in, e.g., nerve-end neuroma, compressed nerves or nerve roots, dorsal root ganglia, and the thalamus may in different conditions underlie the spontaneous pain. Neuropathic pain includes peripheral neuropathic pain that particularly affects the peripheral nerves, meaning the nerves located outside the brain and spinal cord. In particular, neuropathic pain within the meaning of this invention relates to post-surgical neuropathic pain, as well as neuropathic pain associated with postherpetic neuralgia or diabetic peripheral neuropathy of the hands or feet.

In this context, the term “post-surgical neuropathic pain” is understood to mean chronic pain that develops after a surgical procedure and persists beyond the healing process, i.e. at least three months after the surgery. The pain is either localized to the surgical field or area of injury, projected to the innervation territory of a nerve situated in this area, or referred to a dermatome (after surgery/injury to deep somatic or visceral tissues). Chronic post-surgical pain is the result of nerve damage and can be due to the surgery itself or other causes of pain including infection, malignancy, etc.

In this context, the term “postherpetic neuralgia”, also referred to as post-shingles nerve pain, is understood to mean pain occurring if nerves are damaged due to a previous herpes zoster infection, commonly referred to as shingles. Symptoms of post-shingles nerve pain are often limited or localized to the area of the skin where the shingles outbreak first occurred, in the band around the trunk, usually on one side of the body. Less common symptoms of post-shingles nerve pain include itching, numbness, or feeling “pins and needles.”

In this context, the term “diabetic peripheral neuropathy”, also referred to as diabetic nerve pain”, is understood to mean pain occurring if nerves are damaged as a result of diabetes. Although diabetic nerve pain can affect any nerve, it is most often felt in extremities, such as the hands or feet.

Within the meaning of this invention, the term “joint pain” refers to an articular condition, such as discomfort, aches or soreness in any of the body's joints of a patient, including spine, shoulders, hips, elbows and knees. This includes in particular joint pain caused by arthritis, such as osteoarthritis. In this context, the term “osteoarthritis” is understood to mean a degenerative disease characterized by cartilage erosion, bony hypertrophy, subchondral sclerosis, and synovial and capsular changes. It is clinically characterized by joint pain, stiffness, and functional limitation. Although the pain of osteoarthritis is traditionally considered to be nociceptive, some patients also have neuropathic pain. The joint pain may be in particular knee pain, elbow pain, hip pain, shoulder pain, pain of the hands or feet, or pain of the (lower) back.

Within the meaning of this invention, the term “cancer pain” relates to neuropathic cancer pain caused by nerve damage attributable to the cancer per se, and/or treatments including chemotherapy, radiotherapy, and surgery. Cancer pain caused by the tumor per se usually involves both nociceptive and neuropathic components, and mixed pain is more common than neuropathic cancer pain caused by cancer treatments. Most cancer pain caused by chemotherapy is purely neuropathic in nature. Neuropathic cancer pain is nerve-related (typically neuron-related) pain characterized as a burning or electrical sensation; however, it sometimes manifests as decreased sensation or actual muscle weakness.

Within the meaning of the invention, the term “coating composition” refers to a composition comprising all components of the active layer or the skin contact layer, respectively, which may be coated onto the backing layer or release liner to form the active layer and the skin contact layer upon drying.

Within the meaning of this invention, the term “dissolve” refers to the process of obtaining a solution, which is clear and does not contain any particles, as visible to the naked eye.

Within the meaning of this invention, the term “cross-linking” refers to the process of cross-link functional groups contained within the active-free coating composition.

Within the meaning of this invention, and unless otherwise specified, the term “about” refers to an amount that is ±10% of the disclosed amount. In some embodiments, the term “about” refers to an amount that is ±5% of the disclosed amount. In some embodiments, the term “about” refers to an amount that is ±2% of the disclosed amount.

### BRIEF DESCRIPTION OF THE DRAWINGS

FIG. 1a depicts a hexagonal tiling with regular hexagons.

FIG. 1b depicts a hexagonal tiling with parallelogons obtained by elongating rhombuses.

FIG. 1c depicts a hexagonal tiling with parallelogons obtained by elongating parallelograms.

FIG. 2a depicts an exemplary pattern for a sheet of medical patches according to the invention, in which the self-adhesive layer structures are regular hexagons.

FIG. 2b depicts an exemplary pattern for a sheet of medical patches according to the invention, in which the self-adhesive layer structures are double-hexagons formed of two identical regular hexagons sharing two adjacent vertices and their common side.

FIG. 2c depicts an exemplary pattern for a sheet of medical patches according to the invention, in which the self-adhesive layer structures are selected from regular hexagons and double-hexagons formed of two identical regular hexagons sharing two adjacent vertices and their common side.

FIG. 3 depicts a section of an exemplary pattern for a sheet of medical patches according to the invention, in which the self-adhesive layer structures are regular hexagons that are connected to each other by common fastening bridges, wherein neighboring self-adhesive layer structures are all connected to each other in threes by a common fastening bridge provided at the common vertex.

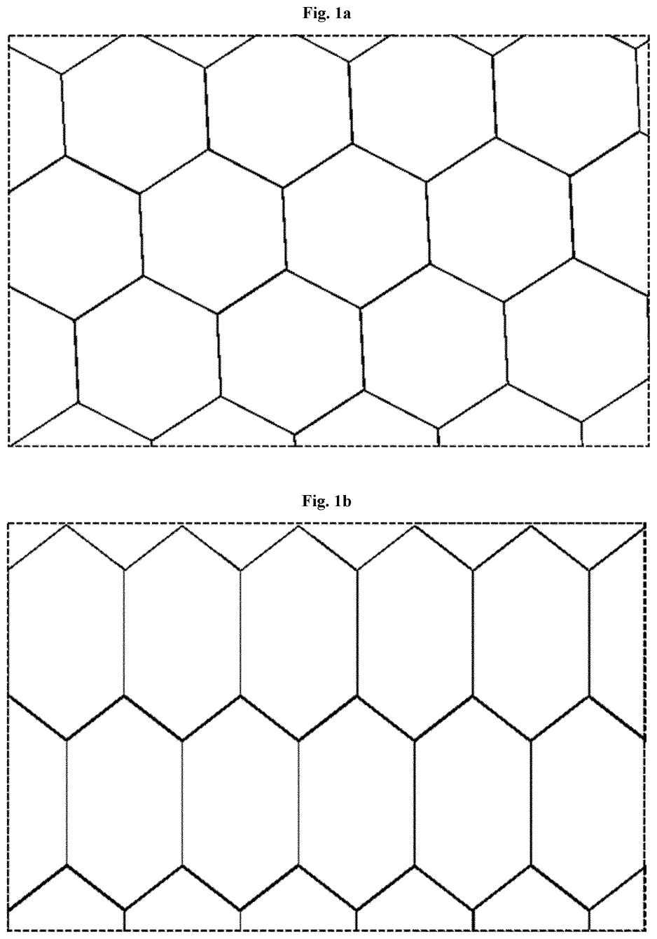

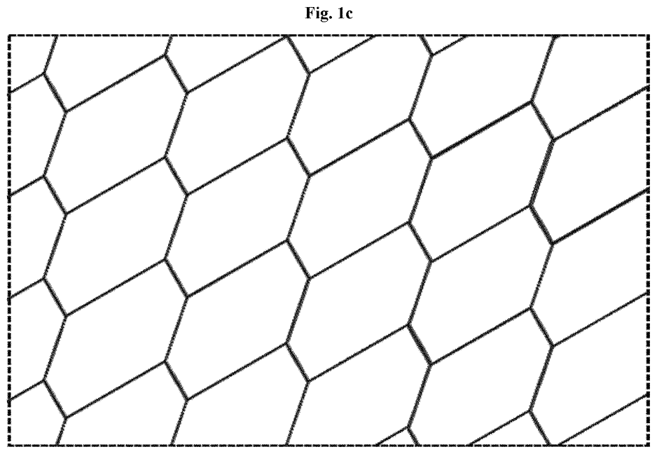

### DETAILED DESCRIPTION

### Self-Adhesive Layer Structure

The present invention relates to a self-adhesive layer structure for use in a medical patch in particular for the administration of an active agent contained therein. In certain embodiments, the self-adhesive layer structure is a pressure sensitive adhesive layer structure.

The self-adhesive layer structure according to the present invention, in particular the pressure sensitive adhesive layer structure, has a hexagonal shape and comprises:
A) a backing layer; and
B) an active layer comprising a polymer I and an active agent;
wherein the backing layer and the active layer are coextensive and provide the hexagonal shape of the self-adhesive layer structure.

In certain embodiments, the self-adhesive layer structure comprises
A) a backing layer;
B) an active layer comprising a polymer I and an active agent; and
C) a skin contact layer;
wherein the backing layer, the active layer and the skin contact layer are coextensive and provide the hexagonal shape of the self-adhesive layer structure.
   In such embodiment, the self-adhesive layer structure may or may not comprise a membrane which is located between the active layer and the skin contact layer. The membrane preferably is a rate controlling membrane.

In certain embodiments, the aforementioned layers of the self-adhesive layer structure according to the invention are directly attached to each other, i.e. the backing layer is directly attached to the active layer, and optionally the active layer is directly attached to an additional skin contact layer. Alternatively, the active layer is directly attached to a membrane, which is on the other side directly attached to the additional skin contact layer. In other words, the self-adhesive layer structure according to the present invention comprises its layers in the following order: (1) backing layer, (2) active layer, and optionally (3) skin contact layer, or (1) backing layer, (2) active layer, optionally (3) membrane, and optionally (4) skin contact layer.

The optional additional skin contact layer preferably provides for adhesion between the self-adhesive layer structure and the skin of the patient during administration. If the self-adhesive layer structure according to the invention does not comprise an additional skin contact layer, sufficient adhesion between the self-adhesive layer structure and the skin of the patient during administration is provided for by other means, e.g. the active layer and/or an adhesive overlay.

The backing layer is in particular substantially impermeable to the active agent. It may consist of a polyester film, preferably with a thickness of 10-20 μm, or of an ethylene-vinyl acetate copolymer.

In certain embodiments, the self-adhesive layer structure is for transdermal or topical delivery of the active agent. In particular embodiments, the self-adhesive layer structure is for topical delivery of the active agent.

The self-adhesive layer structure according to the present invention may be used in a matrix-type medical patch or a reservoir-type medical patch, and preferably is a matrix-type medical patch. In certain embodiments, the self-adhesive layer structure according to the present invention is for use in a matrix-type medical patch, wherein the active agent is homogeneously dissolved and/or dispersed within a polymeric carrier, i.e. the matrix, which forms together with the active agent and optionally further additives a matrix layer. Accordingly, it is preferred for the active layer to be an active matrix layer. Thus, in certain embodiments of the self-adhesive layers structure according to the present invention, the active layer is an active matrix layer comprising
(i) the polymer I, and
(ii) the active agent.

In particular embodiments, the self-adhesive layer structure according to the present invention is for use in a microreservoir-type medical patch. Accordingly, it is preferred for the active layer to be a microreservoir active layer, in particular a dried biphasic layer having
(i) an outer phase comprising the polymer I, and
(ii) an inner phase comprising the active agent,
wherein the inner phase forms dispersed deposits in the outer phase.

The self-adhesive layer structure according to the invention is normally located on a detachable protective layer (release liner) from which it is removed immediately before application to the surface of the patient's skin. Thus, the self-adhesive layer structure or the medical patch may further comprise a release liner. A self-adhesive layer structure or a medical patch protected this way is usually stored in a seam-sealed pouch. The packaging may be child resistant and/or senior friendly.

### Active Layer

As outlined in more detail above, the self-adhesive layer structure according to the present invention comprises inter alia an active layer, which comprises
(i) a polymer I, and
(ii) an active agent.

The active agent is preferably homogeneously distributed within the active layer. In particular embodiments, the active layer is an active matrix layer, in particular a microreservoir active layer.

Thus, in certain embodiments of the self-adhesive layer structure, the active layer is a dried biphasic layer having
(i) an outer phase comprising the polymer I, and
(ii) an inner phase comprising the active agent,
wherein the inner phase forms dispersed deposits in the outer phase.
   In certain embodiments, the outer phase is hydrophobic and the inner phase is hydrophilic.

The outer phase of the dried biphasic layer preferably has a composition comprising 75% to 100% of the polymer I. The inner phase preferably has a composition comprising the active agent and a hydrophilic agent forming a solution with the active agent. The hydrophilic agent may be a hydrophilic polymer or polymer mixture, which is in particular selected from the group consisting of polyvinylpyrrolidones having a K-Value of from 10 to 200, copolymers of vinyl caprolactam, vinylacetate and ethylene glycol, copolymers of vinylpyrrolidone and vinylacetate, copolymers of ethylene and vinylacetate, polyethylene glycols, polypropylene glycols, acrylic polymers, and modified celluloses.

The dried biphasic layer may further comprise an interface mediator in particular with a kinematic viscosity of from 10 cSt to 100 000 cSt at 25° C. The interface mediator may be present in the dried biphasic layer in an amount of 0.1% to 3.5%, and is used to reduce the maximum droplet size of the dispersed deposits of the inner phase in the outer phase in the dried biphasic layer. Without wishing to be bound to any theory it is believed that this effect is achieved by filling cavities at the interface between the dispersed inner phase and the outer phased and thereby enhancing the compatibility of the two separate phases and promoting a maximum separation/dispersion of the inner phase in the outer phase. Suitable interface mediators include, e.g., silicone oil.

The polymer I contained in the active layer provides for sufficient cohesion of the active layer. According to certain embodiments, the polymer I may also provide for sufficient adhesion of the self-adhesive layer structure to the skin of the patient during administration. In those embodiments, the polymer I is selected from pressure sensitive adhesive polymers. Thus, in certain embodiments, the polymer I is a pressure sensitive adhesive polymer.

Polymers which are suitable as the polymer I in accordance with the invention may be selected from silicone-based polymers, acrylic polymers, silicone acrylic hybrid polymers, and polymers based on natural or synthetic rubber, such as polyisobutylenes or styrene-isoprene-styrene block copolymers, which are described in more detail below. In certain embodiments, the polymer is selected from silicone-based polymers. In particular embodiments, the polymer is a silicone-based polymer obtainable by polycondensation of silanol endblocked polydimethylsiloxane with a silicate resin.

Further, in certain embodiments, the area weight of the active layer ranges from 20 to 400 g/m², from 30 to 200 g/m², or from 50 to 120 g/m².

### Active Agent

In accordance with the invention, the self-adhesive layer structure comprises an active layer comprising an active agent.

The active agent may be any compound responsible for the therapeutic effect(s) of the medical patch containing the self-adhesive layer structure. In particular, the active agent may be a topically active agent or a systemically active agent. In certain embodiments, the active agent is at least one analgesic. Suitable analgesics include, for example, buprenorphine, capsaicin, diclophenac, fentanyl, ibuprofen or lidocaine.

According to certain embodiments, the active agent is a TRPV1 agonist, such as capsaicin.

In one embodiment, the active agent is capsaicin. Thus, the self-adhesive layer structure may comprise
A) a backing layer; and
B) an active layer comprising
   (i) a polymer I, and
   (ii) capsaicin.

In particular, the self-adhesive layer structure comprises capsaicin in a therapeutically effective amount. In certain embodiments, the self-adhesive layer structure comprises the capsaicin in an amount of from 0.5 to 180 mg, from 1.2 to 90 mg, or 19 to 45 mg. In some embodiments, the self-adhesive layer structure comprises the capsaicin in an amount of about 179 mg. In other embodiments, the self-adhesive layer structure comprises the capsaicin in an amount of about 60 mg, of about 45 mg, of about 30 mg, of about 25 mg, of about 10 mg, or of about 1 mg.

Particular active layers according to the invention thus comprise
(i) a polymer I, and
(ii) capsaicin.

The active layer may contain at least 0.30 mg/cm², at least 0.50 mg/cm², or at least 0.60 mg/cm² capsaicin per area of release, and/or less than 1.0 mg/cm², less than 0.8 mg/cm², or less than 0.7 mg/cm² capsaicin per area of release. In particular, the active layer contains from 0.30 mg/cm² to 1.0 mg/cm², from 0.30 mg/cm² to 8.0 mg/cm2, from 0.50 mg/cm² to 8.0 mg/cm², from 0.60 mg/cm² to 0.8 mg/cm², or from 0.60 mg/cm² to 0.7 mg/cm² capsaicin per area of release.

In certain embodiments, the active layer comprises the capsaicin in an amount of from 2 to 20% by weight, from 5 to 15% by weight, or from 5 to 10% by weight. In particular embodiments, the active layer comprises the capsaicin in an amount of about 8% by weight.

Further, the active layer may comprise at least one silicone-based polymer in an amount of from 20 to 90% by weight, or 60 to 90% by weight, based on the total weight of the active layer. It is to be understood that the aforementioned weight percent amounts refer to the overall amount of the at least one silicone-based polymer. For example, if two silicone-based polymers are present, the overall amount in the active layer is from 20 to 90% by weight, or 60 to 90% by weight, based on the total weight of the active layer.

Thus, in one embodiment, the self-adhesive layer structure is for transdermal or topical delivery of capsaicin, in particular for topical delivery of capsaicin.

Capsaicin ((6E)-N-[(4-hydroxy-3-methoxyphenyl)methyl]-8-methylnon-6-enamide) is the major bioactive component of chili peppers, and an alkaloid found in the Capsicum family. It is a potent agonist of the transient receptor potential cation channel subfamily V member 1 (TRPV1), better known as vanilloid receptor. By binding to the TRPV1 receptor, the capsaicin molecule produces similar sensations to those of excessive heat or abrasive damage. Capsaicin has a role as a non-narcotic analgesic and is currently used for treatment of several pain syndromes such as neuropathic pain. Such pain is believed to result from sensitization reactions in the peripheral and central nervous system and can occur as a result of peripheral injuries, or as a result of system diseases such as HIV, herpes zoster syphilis, autoimmune diseases and diabetes. Furthermore, capsaicin has also demonstrated beneficial effect on osteoarthritis pain relief due to its high capacity to inhibit P substance release, a powerful neuropeptide pain neuromodulator from the sensory nerves to the central nervous system. In addition, capsaicin is suggested to be able to kill cancer cells by causing them to undergo apoptosis.

The active agent, in particular the capsaicin may be present in the active layer in an amount of from 1 to 25% by weight, from 2 to 20% by weight or from 5 to 10% by weight.

### Skin Contact Layer

As outlined in more detail above, the self-adhesive layer structure according to the present invention may additionally comprise a skin contact layer. In this case, the backing layer and the active layer as well as the skin contact layer are coextensive and provide the hexagonal shape of the self-adhesive layer structure. In certain embodiments, the skin contact layer is adhesive, in particular pressure sensitive adhesive, and provides for adhesion between the self-adhesive layer structure and the skin of the patent during administration.

It has been surprisingly found that a self-adhesive layer structure comprising a skin contact layer, in which the active agent is sparingly soluble and which is preferably directly attached to an active layer, has advantageous properties regarding reduced skin irritation, but at the same time has improved drug delivery behavior and adhesive properties. In particular, since the active agent is sparingly soluble, e.g., has a saturation concentration of less than 0.1% in the skin contact layer, only an insignificant amount of the active agent is present on the surface of the skin contact layer so that the medical patch has advantageous properties regarding unwanted skin reactions and thus enables safe application and/or removal. Thus, such self-adhesive layer structure with a skin contact layer, in which the saturation concentration of the active agent is negligible, with a separated active layer (not being in skin contact), prohibits the active agent to be released before and/or after the self-adhesive layer structure is applied to and maintained on the skin of the patient. On the other hand, it has been found that such a self-adhesive layer structure with a skin contact layer is still able to provide a sufficient drug delivery and even allows for a faster release of active.

Accordingly, such a self-adhesive layer structure comprising a skin contact layer is in particular characterized by the low solubility of the active agent in the skin contact layer, and in certain embodiments, the saturation concentration of the capsaicin in the skin contact layer is less than 0.1% by weight, preferably as determined by the “sandwich method”. In certain embodiments, the saturation concentration of the active agent in the skin contact layer is less than 0.05% by weight, less than 0.02% by weight, or less than 0.01% by weight. Preferably, the saturation concentration of the active agent in the skin contact layer is about 0% by weight. The saturation concentration relates to the amount of active agent being present in the skin contact layer, based on the total weight of the skin contact layer.

In certain embodiments, the saturation concentration of the active agent in the skin contact layer is less than a concentration of the active agent resulting in any unintended adverse effect such as a skin irritation upon contact after a short period of time. Such a concentration may be determined empirically by in vivo testing, by observing whether an adverse effect such as any form of skin irritation (redness, erythema, itching or other skin reaction) occurs or does not occur after applying model adhesive layers with defined capsaicin concentrations on the skin for a short period of time, e.g., 5 seconds, 10 seconds, 30 seconds or 1 minute. In particular, different model layers representing a series of active agent concentrations may be tested to determine the highest acceptable saturation concentration which will not yet cause any unintended adverse effect such as a skin irritation reaction. On the other hand, whether or not a medical patch with a certain set of active agent and skin contact layer results in a saturation concentration not resulting in any adverse effect can be simply determined (without a series of different concentration) by testing a model adhesive layer which is saturated with the active agent, or said medical patch, i.e., by applying on the skin as outlined above.

The skin contact layer may shield the active agent contained in the active layer from the skin of the patient or other applying/removing person before and/or after application of the medical patch. The skin contact layer thus needs to be substantially free of the active agent. This means that the skin contact layer is typically manufactured as a layer free of the active agent. However, due to the concentration gradient, the active agent usually may migrate from the active layer to the skin contact layer over time, until an equilibrium is reached. This migration is, however, limited by the saturation concentration of the active agent in the skin contact layer. In certain embodiments, the skin contact layer does not allow the active agent to be present at a concentration of more than 0.1% by weight.

Thus, in certain embodiments, the skin contact layer comprises the active agent in an amount of less than 0.1% by weight, based on the total weight of the skin contact layer. In particular embodiments, the skin contact layer comprises the active agent in an amount of less than 0.01% by weight, based on the total weight of the skin contact layer.

According to certain embodiments, the skin contact layer comprises a polymer II. The polymer II in the skin contact layer is decisive for the adhesive properties and further reduces skin irritation inter alia due to its resiliency. In certain embodiments the skin contact layer comprises the polymer II in an amount of at least 95% by weight, at least 99% by weight, or in an amount of about 100% by weight, based on the total weight of the skin contact layer. In particular, the skin contact layer may essentially consist of the polymer II. It is to be understood that the aforementioned weight percent amounts refer to the overall amount of the polymer II. For example, if the polymer II is a mixture of polymers, the overall amount in the skin contact layer is from 50 to 100% by weight, based on the total weight of the skin contact layer.

Polymers which are suitable as the polymer II in accordance with the invention are in particular polymers allowing the active agent to concentrate to not more than 0.1% by weight, not more than 0.05% by weight, not more than 0.02% by weight, or not more than 0.01% by weight, i.e. polymers in which the active agent is substantially insoluble. Thus, according to certain embodiments, the polymer II may be a polymer or a mixture of polymers in which the active agent is substantially insoluble.

Accordingly, the solubility parameter of the polymer II may differ from, in particular may be lower than the solubility parameter of the active agent by at least 5.0 MPa^{1/2}, at least 6.0 MPa^{1/2}, at least 8.0 MPa^{1/2}, or at least 10.0 MPa^{1/2}. In particular, the solubility parameter of the polymer II may be less than 18.5 MPa^{1/2}, less than 18.0 MPa^{1/2}, less than 17.5 MPa^{1/2}, less than 17.0 MPa^{1/2}, less than 16.0 MPa^{1/2}, or less than 15.0 MPa^{1/2}, preferably as calculated by Small's method.

The polymer II may be selected from pressure sensitive adhesive polymers. Thus, in certain embodiments, the polymer II may be a pressure sensitive adhesive or a mixture of pressure sensitive adhesives.

In certain embodiments, the polymer II may be a polymer or a mixture of polymers selected from the group consisting of silicone acrylic hybrid polymers, silicone-based polymers, silicone gel adhesives, and polymers based on natural or synthetic rubbers, which are described in more detail below. In particular, the polymer II may be a polymer or a mixture of polymers selected from the group consisting of silicone-based polymers and silicone gel adhesives.

In particular embodiments, the polymer II may be a silicone gel adhesive. Such a self-adhesive layer structure, which comprises an additional skin contact layer comprising a silicone gel adhesive, when applied to a patient's skin, provides improved wearing properties as well as a clean and painless removal. If necessary, e.g. in the case of repositioning, the self-adhesive layer structure can be removed and applied again without loss of the adhesiveness.

Also, the polymer II may be polymer or a mixture of polymers selected from silicone-based polymers, in particular from polymers based on polysiloxanes such as an amine-compatible polysiloxanes, or the polymer II may be a polymer or a mixture of polymers selected from natural or synthetic rubbers, in particular from styrenic triblock copolymers and/or polyisobutylenes, such as an SIS block copolymer and/or polyisobutylene.

Polymers suitable as a polymer II are commercially available e.g. under the brand names Soft skin adhesives (two-part silicone adhesive that cures upon mixing the two components). Alternatively, polymers suitable as a polymer II are commercially available e.g. under the brand names BIO-PSA (pressure sensitive adhesives based on polysiloxanes) JSR-SIS (SIS block copolymer-based pressure-sensitive adhesives) and Oppanol™ (polyisobutylenes).

Additional polymers may also be added, e.g., to enhance adhesion of the skin contact layer.

According to some embodiments, the polymer II contained in the skin contact layer is different from the polymer I contained in the active layer. According to other embodiments, the polymer II contained in the skin contact layer is the same as the polymer I contained in the active layer.

According to certain embodiments, the area weight of the skin contact layer may range from 80 to 500 g/m². In certain embodiments, the skin contact layer may have an area weight of from 100 to 350 g/m², from 150 to 320 g/m², or from 180 to 280 g/m².

### Silicone-Based Polymer

Suitable silicone-based polymers are non-curing polymers, which are typically applied by a hot-melt or a solvent based process and preferably does not undergo further curing to solidify.

Silicone-based polymers are based on polysiloxanes. They may therefore also be referred to as polymers based on polysiloxanes. Silicone-based polymers are generally obtainable by polycondensation of silanol endblocked polydimethylsiloxane with a silicate resin. Amine-compatible silicone-based polymers can be obtained by reacting the silicone-based polymer with trimethylsilyl (e.g. hexamethyldisilazane) in order to reduce the silanol content of the polymer and thus provide enhanced stability in the presence of amines. As a result, the residual silanol functionality is at least partly, preferably mostly or fully capped with trimethylsiloxy groups.

Thus, in certain embodiments, the silicone-based polymer is an amine-compatible polysiloxane, and preferably is obtainable by polycondensation of a silanol endblocked polydimethylsiloxane with a silicate resin followed by at least partial trimethylsilylation of the residual silanol functionality.

In certain embodiments, the silicone-based polymer is pressure sensitive adhesive or a mixture of pressure sensitive adhesives, i.e. a pressure sensitive adhesive based on polysiloxanes or a mixture of pressure sensitive adhesives based on polysiloxanes

Pressure sensitive adhesives based on polysiloxanes provide for suitable tack and for quick bonding to various skin types, including wet skin, suitable adhesive and cohesive qualities, long lasting adhesion to the skin, a high degree of flexibility, a permeability to moisture, and compatibility to many actives and film-substrates. Such pressure sensitive adhesives are based on a resin-in-polymer concept wherein, by condensation reaction of silanol endblocked polydimethylsiloxane with a silica resin (also referred to as silicate resin), a pressure sensitive adhesive based on polysiloxane is prepared. For amine stability, the residual silanol functionality is additionally capped with trimethylsiloxy groups. The silanol endblocked polydimethylsiloxane content contributes to the viscous component of the visco-elastic behavior, and impacts the wetting and the spreadability properties of the adhesive. The resin acts as a tackifying and reinforcing agent, and participates in the elastic component. The correct balance between silanol endblocked polydimethylsiloxane and resin provides for the correct adhesive properties.

As indicated before, the tackiness of the silicone-based polymer may be modified by the resin-to-polymer ratio, i.e. the ratio of the silanol endblocked polydimethylsiloxane to the silicate resin, which is preferably in the range of from 50:50 to 70:30, or from 55:45 to 65:35. The tackiness will be increased with increasing amounts of the polydimethylsiloxane relative to the resin. High tack silicone-based polymers preferably have a resin-to-polymer ratio of 55:45, medium tack silicone-based polymers preferably have a resin-to-polymer ratio of 60:40, and low tack silicone-based polymers preferably have a resin-to-polymer ratio of 65:35.

According to certain embodiments, the pressure sensitive adhesive is obtainable by polycondensation of a silanol endblocked polydimethylsiloxane with a silicate resin, preferably with a resin-to-polymer ratio of from 50:50 to 70:30, or of 55:45, 60:40 or 65:35. Thus, in one embodiment, the silicone-based polymer is a mixture of pressure sensitive adhesives obtainable by polycondensation of a silanol endblocked polydimethylsiloxane with a silicate resin with a resin-to-polymer ratio of 55:45 or of 60:40.

Further, according to certain embodiments, the silicone-based polymer is a mixture of pressure sensitive adhesives with
a solution viscosity at 25° C. and about 60% solids content in heptane of 450 mPa s and/or a complex viscosity at 0.01 rad/s at 30° C. of 1×10⁸ Poise, and
a solution viscosity at 25° C. and about 60% solids content in heptane of 500 mPa s and/or a complex viscosity at 0.01 rad/s at 30° C. of 5×10⁶ Poise.

The pressure sensitive adhesives based on polysiloxanes are supplied and used in solvents like n-heptane, ethyl acetate or other volatile silicone fluids. The solids content of pressure sensitive adhesives based on polysiloxanes in solvents is usually between 60 and 85% between 70 and 80% or between 60 and 75%. The skilled person is aware that the solids content may be modified by adding a suitable amount of solvent.

High tack silicone-based polymers preferably have a complex viscosity at 0.01 rad/s and 30° C. of about 5×10⁶ Poise, medium tack silicone-based polymers preferably have a complex viscosity at 0.01 rad/s and 30° C. of about 5×10⁷ Poise, and low tack silicone-based polymers preferably have a complex viscosity at 0.01 rad/s and 30° C. of about 5×10⁸ Poise. High tack amine-compatible silicone-based polymers preferably have a complex viscosity at 0.01 rad/s and 30° C. of about 5×10⁶ Poise, medium tack amine-compatible silicone-based polymers preferably have a complex viscosity at 0.01 rad/s and 30° C. of about 5×10⁸ Poise, and low tack amine-compatible silicone-based polymers preferably have a complex viscosity at 0.01 rad/s and 30° C. of about 5×10⁹ Poise. Preferred pressure sensitive adhesives based on polysiloxanes in accordance with the invention are characterized by a solution viscosity at 25° C. and 60% solids content in n-heptane of more than about 150 mPa s, or from about 200 mPa s to about 700 mPa s, preferably as measured using a Brookfield RVT viscometer equipped with a spindle number 5 at 50 rpm. Theses may also be characterized by a complex viscosity at 0.01 rad/s at 30° C. of less than about 1×10⁹ Poise or from about 1×10⁵ to about 9×10⁸ Poise.

Suitable silicone-based polymers are commercially available under the brand names BIO-PSAs. Examples of silicone-based PSA compositions which are commercially available include the standard Liveo™ BIO-PSA series (7-4400, 7-4500 and 7-4600 series) and the amine compatible (endcapped) Liveo™ BIO-PSA series (7-4100, 7-4200 and 7-4300 series) manufactured and typically supplied in n-heptane or ethyl acetate. For example, BIO-PSA 7-4201 is characterized by a solution viscosity at 25° C. and about 60% solids content in heptane of 450 mPa s and a complex viscosity at 0.01 rad/s at 30° C. of 1×10⁸ Poise. BIO-PSA 7-4301 has a solution viscosity at 25° C. and about 60% solids content in heptane of 500 mPa s and a complex viscosity at 0.01 rad/s at 30° C. of 5×10⁶ Poise.

Pressure sensitive adhesives based on polysiloxanes may be obtained according to the following scheme:

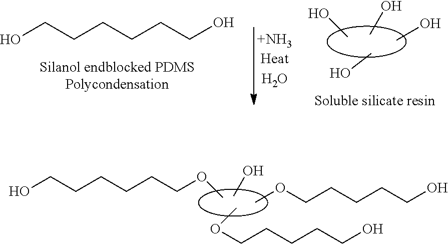

Such pressure sensitive adhesives based on polysiloxanes are available under the tradenames Liveo™ BIO-PSA 7-4401, BIO-PSA-7-4501, or BIO-PSA 7-4601, which are provided in the solvent n-heptane (indicated by the code “01”), or under the tradenames Liveo™ BIO-PSA 7-4402, BIO-PSA 7-4502, and BIO 7-4602, which are provided in the solvent ethyl acetate (indicated by the code “02”). Typical solids contents in the solvent are in the range of from 60 to 75%. The code “44” indicates a resin-to-polymer ratio of 65:35 resulting in a low tackiness, the code “45” indicates a resin-to-polymer ratio of 60:40 resulting in medium tackiness, the code “46” indicates a resin-to-polymer ratio of 55:45 resulting in high tackiness.

Amine-compatible pressure sensitive adhesives based on polysiloxanes may be obtained according to the following scheme:

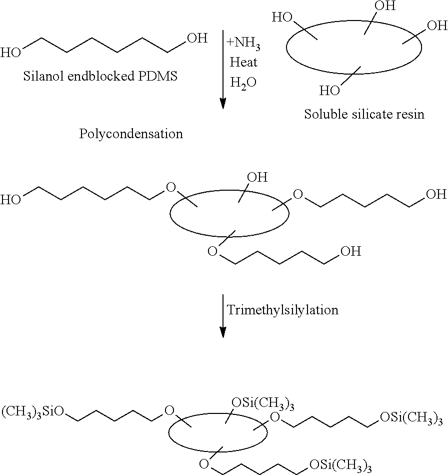

Such amine-compatible pressure sensitive adhesives based on polysiloxanes are available under the tradenames Liveo™ BIO-PSA 7-4101, BIO-PSA-7-4201, or BIO-PSA 7-4301, which are provided in the solvent n-heptane (indicated by the code “01”), or under the tradenames Liveo™ BIO-PSA 7-4102, BIO-PSA 7-4202, and BIO 7-4302, which are provided in the solvent ethyl acetate (indicated by the code “02”). Typical solids contents in the solvent are in the range of from 60 to 75%. The code “41” indicates a resin-to-polymer ratio of 65:35 resulting in a low tackiness, the code “42” indicates a resin-to-polymer ratio of 60:40 resulting in medium tackiness, the code “43” indicates a resin-to-polymer ratio of 55:45 resulting in high tackiness.

### Acrylic Polymer

As used herein, the terms acrylic polymer and acrylate polymer are synonymously referred to polymers based on acrylates. According to certain embodiments, the acrylic polymers are pressure-sensitive adhesives based on acrylates. Pressure-sensitive adhesives based on acrylates may also be referred to as acrylate-based pressure-sensitive adhesives, or acrylate pressure-sensitive adhesives.

Pressure-sensitive adhesives based on acrylates may be provided in the form of a solution with a solids content preferably between 30% and 60%. Acrylate-based pressure-sensitive adhesives may or may not comprise functional groups such as hydroxy groups, carboxylic acid groups, neutralized carboxylic acid groups and mixtures thereof. Corresponding commercial products are available e.g. from Henkel under the tradename Duro Tak®. Such acrylate-based pressure-sensitive adhesives are based on monomers selected from one or more of acrylic acid, 2-ethylhexylacrylate, glycidylmethacrylate, 2-hydroxyethylacrylate, methylacrylate, methylmethacrylate, butylacrylate, butylmethacrylate, t-octylacrylamide and vinylacetate, and are provided in ethyl acetate, heptane, n-heptane, hexane, methanol, ethanol, isopropanol, 2,4-pentanedione, toluene or xylene or mixtures thereof.

Specific acrylate-based pressure-sensitive adhesives are commercially available as:
Duro-Tak™ 387-2287 or Duro-Tak™ 87-2287 (a copolymer based on vinyl acetate, 2-ethylhexyl-acrylate, 2-hydroxyethyl-acrylate and glycidyl-methacrylate provided as a solution in ethyl acetate without cross-linking agent),
Duro-Tak™ 387-2516 or Duro-Tak™ 87-2516 (a copolymer based on vinyl acetate, 2-ethylhexyl-acrylate, 2-hydroxyethyl-acrylate and glycidyl-methacrylate provided as a solution in ethyl acetate, ethanol, n-heptane and methanol with a titanium cross-linking agent),
Duro-Tak™ 387-2051 or Duro-Tak™ 87-2051 (a copolymer based on acrylic acid, butylacrylate, 2-ethylhexylacrylate and vinyl acetate, provided as a solution in ethyl acetate and heptane),
Duro-Tak™ 387-2353 or Duro-Tak™ 87-2353 (a copolymer based on acrylic acid, 2-ethylhexylacrylate, glycidylmethacrylate and methylacrylate, provided as a solution in ethyl acetate and hexane),
Duro-Tak™ 87-4098 (a copolymer based on 2-ethylhexyl-acrylate and vinyl acetate, provided as a solution in ethyl acetate).
Duro-Tak™ 387-9301 (a copolymer based on methyl acrylate, 2-ethylhexyl acrylate and t-octyl acrylamide, provided as a solution in ethyl acetate).

Accordingly, the acrylic polymer may be selected from acrylic polymers comprising functional groups wherein the functional groups are selected from hydroxyl groups, carboxylic acid groups, neutralized carboxylic acid groups and mixtures thereof. In certain embodiments, the functional groups are limited to hydroxyl groups. The acrylic polymer may not comprise carboxylic acid groups or neutralized carboxylic acid groups or both groups, or may not comprise acidic groups, or may comprise no functional groups.

Depending on the type of commercially available acrylic polymer used and depending on whether a cross-linking agent is added to the coating composition, the polymer in the finalized active layer or skin contact layer is cross-linked (and preferably is cross-linked by an aluminium and/or a titanium cross-linking agent) or is not cross-linked by a cross-linking agent.

### Silicone Acrylic Hybrid Polymer

As used herein, silicone acrylic hybrid polymers comprise a polymerized hybrid species that includes silicone-based sub-species and acrylate-based sub-species that have been polymerized together. The silicone acrylic hybrid polymer thus comprises a silicone phase and an acrylic phase. According to certain embodiments, the silicone acrylic hybrid polymer is a silicone acrylic hybrid pressure-sensitive adhesive.

Silicone acrylic hybrid pressure-sensitive adhesives are usually supplied and used in solvents like n-heptane and ethyl acetate. The solids content of the pressure-sensitive adhesives is usually between 30% and 80%. The skilled person is aware that the solids content may be modified by adding a suitable amount of solvent.

In certain embodiments, the weight ratio of silicone to acrylate in the silicone acrylic hybrid pressure-sensitive adhesive is from 5:95 to 95:5, or from 20:80 to 80:20, or from 40:60 to 60:40, or the ratio of silicone to acrylate is about 50:50. Suitable silicone acrylic hybrid pressure-sensitive adhesives which are commercially available include the PSA series 7-6100 and 7-6300 manufactured and supplied in n-heptane or ethyl acetate by Dupont (7-610X and 7-630X; X=1 n-heptane-based/X=2 ethyl acetate-based). For example, the 7-6102 silicone acrylic hybrid PSA having a silicone/acrylate ratio of 50/50 is characterized by a solution viscosity at 25° C. and about 50% solids content in ethyl acetate of 2,500 cP and a complex viscosity at 0.1 rad/s at 30° C. of 1.0e7 Poise. The 7-6302 silicone acrylic hybrid PSA having a silicone/acrylate ratio of 50/50 has a solution viscosity at 25° C. and about 50% solids content in ethyl acetate of 1,500 cP and a complex viscosity at 0.1 rad/s at 30° C. of 4.0e6 Poise.

Depending on the solvent in which the silicone acrylic hybrid pressure-sensitive adhesive is supplied, the arrangement of the silicone phase and the acrylic phase providing a silicone or acrylic continuous external phase and a corresponding discontinuous internal phase is different. If the silicone acrylic hybrid pressure-sensitive adhesive is provided in n-heptane, the composition contains a continuous, silicone external phase and a discontinuous, acrylic internal phase. If the silicone acrylic hybrid pressure-sensitive adhesive is provided in ethyl acetate, the composition contains a continuous, acrylic external phase and a discontinuous, silicone internal phase. After evaporating the solvent in which the silicone acrylic hybrid pressure-sensitive adhesive is provided, the phase arrangement of the resulting pressure-sensitive adhesive film or layer corresponds to the phase arrangement of the solvent-containing adhesive coating composition. For example, in the absence of any substance that may induce an inversion of the phase arrangement in a silicone acrylic hybrid pressure sensitive adhesive composition, a pressure-sensitive adhesive layer prepared from a silicone acrylic hybrid pressure-sensitive adhesive in n-heptane provides a continuous, silicone external phase and a discontinuous, acrylic internal phase, a pressure-sensitive adhesive layer prepared from a silicone acrylic hybrid pressure-sensitive adhesive in ethyl acetate provides a continuous, acrylic external phase and a discontinuous, silicone internal phase. The phase arrangement of the compositions can, for example, be determined in peel force tests with pressure-sensitive adhesive films or layers prepared from the silicone acrylic hybrid PSA compositions which are attached to a siliconized release liner. The pressure-sensitive adhesive film contains a continuous, silicone external phase if the siliconized release liner cannot or can only hardly be removed from the pressure-sensitive adhesive film (laminated to a backing film) due to the blocking of the two silicone surfaces. Blocking results from the adherence of two silicone layers which comprise a similar surface energy. The silicone adhesive shows a good spreading on the siliconized liner and therefore can create a good adhesion to the liner. If the siliconized release liner can easily be removed the pressure-sensitive adhesive film contains a continuous, acrylic external phase. The acrylic adhesive has no good spreading due to the different surface energies and thus has a low or almost no adhesion to the siliconized liner.

The silicone acrylic hybrid polymer may be a silicone acrylic hybrid pressure-sensitive adhesive obtainable from a silicone-containing pressure-sensitive adhesive composition comprising acrylate or methacrylate functionality. It is to be understood that the silicone-containing pressure-sensitive adhesive composition comprising acrylate or methacrylate functionality can include only acrylate functionality, only methacrylate functionality, or both acrylate functionality and methacrylate functionality.

The silicone acrylic hybrid pressure-sensitive adhesive may comprise the reaction product of (a) a silicone-containing pressure-sensitive adhesive composition comprising acrylate or methacrylate functionality, (b) an ethylenically unsaturated monomer, and (c) an initiator. That is, the silicone acrylic hybrid pressure-sensitive adhesive is the product of the chemical reaction between these reactants ((a), (b), and (c)). In particular, the silicone acrylic hybrid pressure-sensitive adhesive may include the reaction product of (a) a silicone-containing pressure-sensitive adhesive composition comprising acrylate or methacrylate functionality, (b) a (meth)acrylate monomer, and (c) an initiator (i.e., in the presence of the initiator). That is, the silicone acrylic hybrid pressure-sensitive adhesive may include the product of the chemical reaction between these reactants ((a), (b), and (c)).

The reaction product of (a) a silicone-containing pressure-sensitive adhesive composition comprising acrylate or methacrylate functionality, (b) an ethylenically unsaturated monomer, and (c) an initiator may contain a continuous, silicone external phase and a discontinuous, acrylic internal phase or the reaction product of (a), (b), and (c) may contain a continuous, acrylic external phase and a discontinuous, silicone internal phase.

The silicone acrylic hybrid polymer may comprise a reaction product of a silicone polymer, a silicone resin and an acrylic polymer, wherein the acrylic polymer is covalently self-crosslinked and covalently bound to the silicone polymer and/or the silicone resin.

The silicone acrylic hybrid polymer may comprise a reaction product of a silicone polymer, a silicone resin and an acrylic polymer, wherein the silicone resin contains triorganosiloxy units R₃SiO_{1/2} where R is an organic group, and tetrafunctional siloxy units SiO_{4/2} in a mole ratio of from 0.1 to 0.9 R₃SiO_{1/2} units for each SiO_{4/2}.

The acrylic polymer may comprise at least an alkoxysilyl functional monomer, polysiloxane-containing monomer, halosilyl functional monomer or alkoxy halosilyl functional monomer. In certain embodiments, the acrylic polymer is prepared from alkoxysilyl functional monomers selected from the group consisting of trialkoxylsilyl (meth)acrylates, dialkoxyalkylsilyl (meth)acrylates, and mixtures thereof, or comprises end-capped alkoxysilyl functional groups. The alkoxysilyl functional groups may preferably be selected from the group consisting of trimethoxylsilyl groups, dimethoxymethylsilyl groups, triethoxylsilyl, diethoxymethylsilyl groups and mixtures thereof.

The acrylic polymer may also be prepared from a mixture comprising polysiloxane-containing monomers, preferably from a mixture comprising polydimethylsiloxane mono (meth)acrylate.

The silicone acrylic hybrid polymer may be prepared by a) reacting silicone polymer with silicone resin to form a resultant product, b) reacting the resultant product of a) with an acrylic polymer containing reactive functionality, wherein the components are reacted in an organic solvent.

The silicone acrylic hybrid polymer may be prepared by a) reacting a silicone resin with an acrylic polymer containing reactive functionality to form a resultant product, b) reacting the resultant product of a) with silicone polymer, wherein the components are reacted in an organic solvent.

The silicone acrylic hybrid polymer may be prepared by a) reacting a silicone polymer with an acrylic polymer containing reactive functionality to form a resultant product, b) reacting the resultant product of a) with silicone resin, wherein the components are reacted in an organic solvent.

Further suitable acrylic polymers, silicone resins, and silicone polymers that can be used for chemically reacting together a silicone polymer, a silicone resin and an acrylic polymer to provide a silicone acrylic hybrid polymer in accordance with the previous paragraphs are detailed in WO 2010/124187.

### Polymer Based on Natural or Synthetic Rubbers

Polymers based on natural or synthetic rubbers include hydrocarbon polymers such as (natural and synthetic) polyisoprene, polybutylene and polyisobutylene, styrene/butadiene polymers, styrene-isoprene-styrene block copolymers, butyl rubber, halogen-containing polymers such as polyacrylic-nitrile, polytetrafluoroethylene, polyvinylchloride, polyvinylidene chloride, and polychlorodiene, other copolymers thereof. The polymers may in particular be used in combination with a tackifier as defined below.

According to certain embodiments, the polymer may be a styrenic triblock copolymer selected from the group consisting of styrene-ethylene-styrene (SES) block copolymers, styrene-butadiene-styrene (SBS) block copolymers, styrene-isoprene-styrene (SIS) block copolymers, styrene-ethylene/butylene-styrene (S-EB-S) block copolymers, styrene-ethylene/butylene/propylene-styrene (s-EBS-S) block copolymers, styrene-isoprene/butadiene-styrene (S-IB-S) block copolymers, and mixtures thereof.

In certain embodiments, the polymer may be at least one SIS block copolymer. The at least one SIS block copolymer may consist of three blocks of polystyrene, polyisoprene and polystyrene and in particular has a molecular weight of from about 100,000 to 200,000. In particular embodiments, the SIS block copolymer may comprise blocks of polystyrene and of polyisoprene in a ratio of from about 10:90(%) to about 30:70(%), or in a ratio of about 15:85(%) or about 22:78(%).

In other embodiments, the polymer is at least one polyisobutylene and may be a combination of two different types of polyisobutylenes, in particular a combination of low-molecular weight polyisobutylene and high-molecular weight polyisobutylene. In particular embodiments, the ratio of the low-molecular weight polyisobutylene to the high-molecular weight polyisobutylene is in the range of from 75:25 to 90:10.

Suitable styrene-isoprene-styrene (SIS) block copolymers according to the invention are commercially available, e.g., under the brand names JSR-SIS. Specific SIS block copolymer-based pressure-sensitive adhesives are available under the tradenames JSR-SIS5229 and JSR-SIS5002.

Suitable polyisobutylenes as used herein are available under the tradename Oppanol®. Combinations of high-molecular weight polyisobutylenes (B100/B80) and low-molecular weight polyisobutylenes (B10, B11, B12, B13) may be used. Suitable ratios of low-molecular weight polyisobutylene to high-molecular weight polyisobutylene are in the range of from 100:1 to 1:100, from 95:5 to 40:60, or from 90:10 to 75:25. A particular example for a polyisobutylene combination is B10/B100 in a ratio of 85/15, or B12/B100 in a ratio of 80/20. Oppanol® B100 has a viscosity average molecular weight Mᵥ of 1,110,000, and a weight average molecular weight M_{w} of 1,550,000, and an average molecular weight distribution M_{w}/Mₙ of 2.9. Oppanol® B10 viscosity average molecular weight Mᵥ of 40,000, and a weight average molecular weight M_{w} of 53,000, and an average molecular weight distribution M_{w}/Mᵥ of 3.2. Oppanol® B12 has a viscosity average molecular weight Mv of 55,000, and a weight average molecular weight Mw of 70,000, and an average molecular weight distribution Mw/Mn of 3.2. A suitable polyisobutylene adhesive is also commercially available e.g. under the brand name Duro-Tak™ 87-6908.

### Silicone Gel Adhesive

The silicone gel adhesive is an elastic, jelly-like material formed by lightly crosslinking silicone polymers. Thus, in contrast to the silicone-based polymers as used herein, the silicone gel adhesive is based on a curable gel producing composition. The silicone gel adhesive, when used in the skin contact layer, provides for the adhesiveness of the medical patch to the skin, while at the same time reducing the problem of skin irritation. Furthermore, the drug delivery of the medical patch is not negatively affected, surprisingly the skin permeation behavior is even improved.

Silicone gel adhesives are also referred to as silicone gels and, e.g., described in WO 2011/022199 A2.

The silicone gel adhesive is generally formed from linear or branched silicones having reactive groups thereon. Such reactive groups undergo a crosslinking reaction during curing. Examples of crosslinking reactions include the hydrosilylation reaction in which a silicone having an Si—H reactive group reacts with a silicone having an aliphatic unsaturated reactive group in the presence of a hydrosilylation catalyst. These materials are described, for example in U.S. Pat. Nos. 5,656,279, 5,891,076, EP 0 322 118 and U.S. Pat. No. 4,991,574 which are incorporated herein by reference. An alternative reaction is the condensation cure in which an alkoxy and/or hydroxy containing siloxanes are cured with a catalyst as described in U.S. Pat. No. 4,831,070 which is hereby incorporated by reference.

Typically, the silicone gel adhesive is obtainable by reacting a gel producing composition comprising (i) at least one alkenyl-substituted polydiorganosiloxane, (ii) at least one organosiloxane, which contains silicone-bonded hydrogen atoms, and (iii) at least one catalyst for the reaction of the SiH groups with the Si-alkenyl groups. These compositions cure at normal ambient temperatures, but curing can be expedited by heating to elevated temperatures, e.g., from 40 to 140° C., or by applying UV light.

Suitable alkenyl groups contain from 2 carbon to about 6 carbon atoms and are exemplified by, but not limited to, vinyl, allyl, and hexenyl. The alkenyl groups in this component may be located at terminal, pendant (non-terminal), or both terminal and pendant positions. The remaining silicone-bonded organic groups in the alkenyl-substituted polydiorganosiloxane are independently selected from the group consisting of monovalent hydrocarbon and monovalent halogenated hydrocarbon groups free of aliphatic unsaturation. These groups typically contain from 1 carbon to about 20 carbon atoms, alternatively from 1 carbon to 8 carbon atoms and are exemplified by, but not limited to, alkyl such as methyl, ethyl, propyl, and butyl; aryl such as phenyl; and halogenated alkyl such as 3,3,3-trifluoropropyl. Typically, at least 50 percent of the organic groups in the alkenyl-substituted polydiorganosiloxane are methyl. The structure of the alkenyl-substituted polydiorganosiloxane is typically linear, however, it may contain some branching due to the presence of trifunctional siloxane units. The viscosity of the alkenyl-substituted polydiorganosiloxane can be any desired. For example, it can be >0 mm²/s to 100,000 mm²/s, alternatively 50 mm²/s to 80,000 mm²/s, alternatively 300 mm²/s-3,000 mm²/s.

Methods for preparing the alkenyl-substituted polydiorganosiloxanes (i) of the present invention, such as condensation of the corresponding halosilanes or equilibration of cyclic polydiorganosiloxanes, are well known in the art.

The alkenyl-substituted polydiorganosiloxanes can be used in the gel producing composition in an amount of 10 wt. %-90 wt. % based on the weight of the composition, alternatively 40 wt. %-90 wt. %, alternatively 50 wt. %-80 wt. %. The amount of alkenyl groups present in the alkenyl-substituted polydiorganosiloxane is typically in the range of 0.05 wt. %-1 wt. %, alternatively 0.05 wt. % to 1 wt. % based on the weight of the alkenyl-substituted polydiorganosiloxane.

The organosiloxane containing silicone-bonded hydrogen atoms (ii) are also known in the art as described, for example in U.S. Pat. No. 3,983,298. The hydrogen atoms in this component may be located at terminal, pendant (non-terminal), or both terminal and pendant positions. The remaining silicone-bonded organic groups in this component are independently selected from the group consisting of monovalent hydrocarbon and monovalent halogenated hydrocarbon groups free of aliphatic unsaturation. These groups typically contain from 1 carbon to about 20 carbon atoms, alternatively from 1 carbon to 8 carbon atoms, and are exemplified by, but not limited to, alkyl such as methyl, ethyl, propyl, and butyl; aryl such as phenyl; and halogenated alkyl such as 3,3,3-trifluoropropyl. In one embodiment of the invention, at least 50 percent of the organic groups in the organosiloxane containing silicone-bonded hydrogen atoms are methyl. The structure of the organosiloxane containing silicone-bonded hydrogen atoms is typically linear however; it may contain some branching due to the presence of trifunctional siloxane units. The viscosity of the organosiloxane containing silicone-bonded hydrogen atoms can be any desired. For example, it can be >0 mm²/s to 100,000 mm²/s, alternatively, 5 mm²/s to 500 mm²/s.

Methods of preparing the organosiloxane containing silicone-bonded hydrogen atoms of the present invention by co-hydrolysis of the appropriate chlorosilanes are known in the art; U.S. Pat. No. 2,877,255 to Clark; Japanese Laid Open Patent Application (KOKAI) SHO 62(1987)-39660 to Mogi et al.; and U.S. Pat. Nos. 5,446,185 and 5,493,040 to Cobb et al., which are all hereby incorporated by reference.

The organosiloxanes containing silicone-bonded hydrogen atoms can be used in the gel producing composition in an amount of 1 wt. %-30 wt. % based on the weight of the composition, alternatively 5 wt. %-20 wt. %, and alternatively 5 wt. %-15 wt. %. In one embodiment, the amount of hydrogen group present in the organosiloxane containing silicone-bonded hydrogen atoms is between 0.05 wt. %-1.44 wt. % based on the weight of the organosiloxane containing silicone-bonded hydrogen atoms.

In the gel producing compositions, (i) and (ii) are preferably present such that the ratio of (H as SiH):(Alkenyl as Si-Alkenyl) is generally in the range of 0.1:1 to 10:1.

The hydrosilylation catalyst (iii) promotes the addition reaction of the alkenyl-substituted polydiorganosiloxane with the organosiloxane containing silicone-bonded hydrogen. The hydrosilylation catalyst can be any of the well known hydrosilylation catalysts comprising a platinum group metal, a compound containing a platinum group metal, or a microencapsulated platinum group metal or compound containing same. These platinum group metals include platinum, rhodium, ruthenium, palladium, osmium and iridium. Platinum and platinum compounds are preferred catalysts based on their high activity level in hydrosilylation reactions. One class of platinum catalysts is the complexes of chloroplatinic acid with certain vinyl-containing organosiloxane compounds disclosed by Willig in U.S. Pat. No. 3,419,593, which is hereby incorporated by reference. A specific catalyst of this type is the reaction product of chloroplatinic acid and 1,3-diethenyl-1,1,3,3-tetramethyldisiloxane.

The hydrosilylation catalyst is present in an amount sufficient to cure the composition of the present invention. Typically, the concentration of the catalyst is sufficient to provide from 0.1 ppm to 500 ppm (part per million), alternatively from 1 ppm to 100 ppm, alternatively from 1 ppm to 50 ppm of a platinum group metal, based on the weight of (i) and (ii).

In view of the above, in one embodiment of the invention, the silicone gel adhesive is obtainable by reacting a gel producing composition comprising (i) a copolymer of vinylmethylsiloxane and dimethylsiloxane with (ii) methylhydrogen polysiloxane with trimethylsilyl endgroups in the presence of (iii) a platinum catalyst, wherein preferably (i) and (ii) are present such that the ratio of (H as SiH):(Alkenyl as Si-Alkenyl) is generally in the range of 0.1:1 to 10:1.

An optional ingredient is a hydroxy substituted silicone resin as described in US. Patent Application No. 2007-0202245, herein incorporated by reference. The resin is typically comprised of groups having the formula R³₃SiO_{1/2} (“M” groups) and groups having the formula SiO_{4/2} (“Q” groups) where R³ is a alkyl group having 1 carbon to 6 carbon atoms or alkylene group having 1 carbon to 6 carbon atoms, typically methyl or vinyl. If an alkenyl group is present in the resin, typically the mol-% of R groups present as alkenyl groups is <10 mol-%, alternatively 5 mol-%. The number ratio of M groups to Q groups is typically in the range of 0.6:1 to 4:1, alternatively 0.6:1 to 1.0:1. The silicone resin typically contains 0.1 wt % to 5 wt %, alternatively 1.0 wt % to 5 wt % silicone-bonded hydroxy groups.

The resin can be used in the gel producing composition in an amount of 2 wt. % to 45 wt. %, based on the weight of the gel producing composition and resin; alternatively 5 wt. % to 40 wt. %, alternatively 10 wt. % to 35 wt. %.

Thus, in one embodiment, the silicone gel adhesive is a silicate resin-reinforced silicone gel adhesive that contains from about 2 to about 45% by weight of at least one hydroxyl substituted silicate resin.

According to certain embodiments, the silicone gel adhesive is a 2-component silicone adhesive system that cures upon mixing the two components. An example of such two-part silicone adhesive which is commercially available includes the Liveo™ Soft Skin Adhesives (e.g. MG 7-9700, MG 7-9800, MG 7-9850 and MG 7-9900) provided as a kit including components A and B. It is a platinum-catalyzed, soft, fillerless elastomeric silicone adhesive for adhering medical devices to the skin with medium adhesion force and gentle removal. The two components A and B are preferably mixed in a ratio of 1:1.

The silicone gel adhesive layer can be made by processes known in the art. For example, the gel may be pre-formed (e.g. as a sheet) by molding, calendaring, extruding, spraying, brushing, applying by hand, casting or coating on a substrate such as a liner. Or the silicone gel layer can be made by applying the gel producing composition to a substrate by spraying, coating, bar coating, etc. Once applied to the substrate the gel producing composition is cured to produce the silicone gel adhesive on the substrate.

### Further Additives

The self-adhesive layer structure according to the invention, and in particular the active layer may further comprise at least one additive or excipient. Said additives or excipients are preferably selected from the group consisting of additional polymers, cross-linking agents, crystallization inhibitors, solubilizers, fillers, tackifiers, plasticizers, stabilizers, softeners, substances for skincare, permeation enhancers, pH regulators, and preservatives. Such additives may be present in the active layer in an amount of from 0.001 to 15% by weight, e.g. from 1 to 10% by weight or from 0.01 to 5% by weight, based on the total weight of the active layer. In a certain embodiment, the total amount of all additives is from 0.001% to 25% of the matrix layer composition. Hereinafter, where a range for an amount of a specific additive is given, such a range refers to the amount per individual additive.

It should be noted that in pharmaceutical formulations, the formulation components are categorized according to their physicochemical and physiological properties, and in accordance with their function. This means in particular that a substance or a compound falling into one category is not excluded from falling into another category of formulation component. E.g. a certain polymer can be a crystallization inhibitor but also a tackifier. Some substances may e.g. be a typical softener but at the same time act as a permeation enhancer. The skilled person is able to determine based on his general knowledge in which category or categories of formulation component a certain substance or compound belongs to. In the following, details on the excipients and additives are provided which are, however, not to be understood as being exclusive. Other substances not explicitly listed in the present description may be as well used in accordance with the present invention, and substances and/or compounds explicitly listed for one category of formulation component are not excluded from being used as another formulation component in the sense of the present invention.

In certain embodiments, the active layer may further comprise an additional polymer, wherein preferably the additional polymer is selected from dimethylpolysiloxanes and ethyl cellulose. Dimethylpolysiloxanes, such as, e.g. dimethicone, are preferably used for increasing the adhesiveness of the active layer, while ethylcellulose preferably functions as viscosity-increasing agent. Other additional polymers of particular interests are polymers with an enhanced ability to absorb water, as higher water and/or moisture absorption assists in maintaining/improving the adhesive properties of the self-adhesive layer structure. Thus, the active layer may further comprise at least one additional polymer selected from polymers, which provide for an improved water and/or moisture absorption of the matrix layer. Such polymers are well known in the art. Of those, particularly suitable and preferred are polyvinylpyrrolidones, and in particular soluble polyvinylpyrrolidones. Other polymers in particular reduce the cold flow and are thus also suitable as additional polymer. A polymeric matrix may show a cold flow, since such polymer compositions often exhibit, despite a very high viscosity, the ability to flow very slowly. Thus, during storage, the matrix may flow to a certain extent over the edges of the backing layer. This is a problem with storage stability and can be prohibited by the addition of certain polymers. A basic acrylate polymer (e.g. Eudragit E100 which is a copolymer based on dimethylaminoethyl methacrylate, butyl methacrylate and methyl methacrylate) may e.g. be used to reduce the cold flow. Thus, the active layer may comprise additionally a basic polymer, in particular an amine-functional acrylate as e.g. Eudragit E100. The additional polymer may be present for example in an amount of from 0 to 20% of the active layer, preferably of from 0.5 to 5% or from 5 to 15% of the active layer.

In certain embodiments, the active layer may further comprise a cross-linking agent. The cross-linking agent may be selected from the group consisting of aluminium and titanium cross-linking agents such as aluminium acetylacetonate, titanium acetylacetonate or polybutyltitanate, and preferably is a titanium cross-linking agent. The amount of cross-linking agent may range from 0.005 to 1%, and preferably from 0.01 to 0.1% of the active layer. The active layer may also comprise a polymer which is self-crosslinking, i.e. comprises a cross-linking functional group such as glycidyl groups, which reacts upon heating. Accordingly, the active layer prefearbly comprises a cross-linking agent as above and a self-crosslinking polymer.

In certain embodiments, the active layer may further comprise a crystallization inhibitor. Suitable examples of crystallization inhibitors include polyvinylpyrrolidone, vinyl acetate/vinylpyrrolidone copolymer and cellulose derivatives. The crystallization inhibitor is preferably polyvinylpyrrolidone, more preferably soluble polyvinylpyrrolidone. The crystallization inhibitor may increase the solubility of the active agent or inhibit the crystallization of the active agent. The crystallization inhibitor can be present in an amount of from 0.5 to 10% by weight based on the total weight of the active layer.

In certain embodiments, the active layer may further comprise a solubilizer. The solubilizer preferably improves the solubility of the active agent in the active layer. Preferred solubilizers include, e.g., glycerol-, polyglycerol-, propylene glycol- and polyoxyethylene-esters of medium chain and/or long chain fatty acids, such as glyceryl monolinoleate, medium chain glycerides and medium chain triglycerides, non-ionic solubilisers made by reacting castor oil with ethylene oxide, and any mixtures thereof which may further contain fatty acids or fatty alcohols, cellulose and methylcellulose and derivatives thereof such as hydroxypropylcellulose and hypromellose acetate succinate, various cyclodextrins and derivatives thereof, non-ionic tri-block copolymers having a central hydrophobic chain of polyoxypropylene flanked by two hydrophilic chains of polyoxyethylene known as poloxamers, water-soluble derivatives of vitamin E, pharmaceutical graded or agglomerated spherical isomalt, a polyethylene glycol, polyvinyl acetate and polyvinylcaprolactame-based graft copolymer, also abbreviated as PVAc-PVCap-PEG and known as Soluplus®, purified grades of naturally derived castor oil, of polyethylene glycol 400, of polyoxyethylene sorbitan monooleate (such as polysorbate 80) or of propylene glycols, diethylene glycol monoethyl ether, glucono-delta-lactone, maize and potato starch, as well as any of the below mentioned soluble polyvinylpyrrolidones, but also insoluble/cross-linked polyvinylpyrrolidones such as crospovidones. However, also the permeation enhancers mentioned below can act as solubilizers. Furthermore, also crystallization inhibitors may act as solubilizers.

Fillers such as silica gels, titanium dioxide and zinc oxide may be used in conjunction with the active layer in order to influence certain physical parameters, such as cohesion and bond strength, in the desired way.

In case the active layer is required to have self-adhesive properties and one or more polymers is/are selected which does/do not provide sufficient self-adhesive properties, a tackifier is added. The tackifier may be selected from polyvinylpyrrolidone (which, due to its ability to absorb water, is able to maintain the adhesive properties of the matrix layer and thus can be regarded as a tackifier in a broad sense), triglycerides, polyethylene glycols, dipropylene glycol, resins, resin esters, terpenes and derivatives thereof, ethylene vinyl acetate adhesives, dimethylpolysiloxanes and polybutenes, preferably polyvinylpyrrolidone and more preferably soluble polyvinylpyrrolidone. The tackifier may be present in an amount of from 5 to 15% of the active layer.

In certain embodiments, the active layer may further comprise a softener/plasticizer. Exemplary softeners/plasticizers include linear or branched, saturated or unsaturated alcohols having 6 to 20 carbon atoms, triglycerides and polyethylene glycols.

In certain embodiments, the active layer may further comprise a stabilizer, wherein the stabilizer is preferably selected from tocopherol and ester derivatives thereof and ascorbic acid and ester derivatives thereof. Preferred stabilizers include sodium metabisulfite, ascorbyl esters of fatty acids such as ascorbyl palmitate, ascorbic acid, butylated hydroxytoluene, tocopherol, tocopheryl acetate and tocopheryl linoleate. Also particularly preferred is a combination of tocopherol and ascorbyl palmitate. Where the active layer comprises a stabilizer, the amount of the stabilizer may be from 0.001 to 2% of the active layer.

In certain embodiments, the active layer may further comprise a substance for skincare. Such substances may be used to avoid or reduce skin irritation as detectable by the dermal response score. Suitable substances for skincare include sterol compounds such as cholesterol, dexpanthenol, alpha-bisabolol, and antihistamines.

In certain embodiments, the active layer may further comprise a permeation enhancer. Permeation enhancers are substances, which influence the barrier properties of the stratum corneum in the sense of increasing the capsaicin permeability. Some examples of permeation enhancers are polyhydric alcohols such as dipropylene glycol, propylene glycol, and polyethylene glycol; oils such as olive oil, squalene, and lanolin; fatty ethers such as cetyl ether and oleyl ether, fatty acid esters such as isopropyl myristate; urea and urea derivatives such as allantoin, polar solvents such as dimethyldecylphosphoxide, methylcetylsulfoxide, dimethylaurylamine, dodecyl pyrrolidone, isosorbitol, dimethylacetonide, dimethylsulfoxide, decylmethylsulfoxide, and dimethylformamide, salicylic acid, amino acids, benzyl nicotinate, and higher molecular weight aliphatic surfactants such as lauryl sulfate salts. Other agents include oleic and linoleic acids, ascorbic acid, panthenol, butylated hydroxytoluene, tocopherol, tocopheryl acetate, tocopheryl linoleate, propyl oleate, and isopropyl palmitate. If the active layer further comprises a permeation enhancer, the permeation enhancer is preferably selected from diethylene glycol monoethyl ether (transcutol), diisopropyl adipate, isopropyl myristate, isopropyl palmitate, lauryl lactate, and dimethylpropylene urea. Particularly preferably, the active layer comprises a permeation enhancer selected from diethylene glycol monoethyl ether.

In certain embodiments, the active layer may further comprise a pH regulator. Suitable pH regulators include mild acids and bases including amine derivatives, inorganic alkali derivatives, and polymers with basic or acidic functionality.

In certain embodiments, the active layer may further comprise a preservative. Suitable preservatives include parabens, formaldehyde releasers, isothiazolinones, phenoxyethanol, and organic acids such as benzoic acid, sorbic acid, levulinic acid and anisic acid.

### Hexagonal Shape

In accordance with the invention, the self-adhesive layer structure has a hexagonal shape which is provided by the backing layer and the active layer, or the backing layer, the active layer and the additional skin contact layer respectively. The hexagonal shape comprises at least one hexagon, wherein
all pairs of opposite sides of the hexagon are parallel, and
the sides of the hexagon have a length of from 0.2 to 10 cm.

The hexagonal shape may comprise one to ten, such as one, two, three, four, or five hexagons, wherein preferably the hexagons adjoin each other and/or do not overlap. The two or more hexagons are preferably integrally connected to each other. Preferred hexagonal shapes do not comprise perforations.

In certain embodiments, the at least one hexagon is at least one convex hexagon, and the hexagonal shape comprises at least one convex hexagon, wherein all pairs of opposite sides of the convex hexagon are parallel, and the sides of the convex hexagon have a length of from 0.2 to 10 cm. In certain embodiments, the hexagonal shape comprises one or two convex hexagons, in particularly integrally connected to each other. In particular embodiments, the hexagonal shape is a convex hexagon.

The hexagonal shape of the self-adhesive layer structure is decisive for easy and less time-consuming handling of a medical patch containing the self-adhesive layer structure. It allows for simplified coverage of skin areas without cutting before application, thus reducing the risk of contaminating the cutting tool or the fingers with active, and also the risk of contaminating the patch at the cut. Further, uneven or rounded skin surfaces may be covered without wrinkling, thus providing full adhesiveness, and even complicated areas, such as fingers or toes may be easily surrounded using the self-adhesive layer structure. In particular, where the hexagonal shape comprises at least one convex hexagon, the hexagonal shape only requires a short side length in relation to the area provided, thus reducing the risk of detaching edges of the medical patch.

In certain embodiments, the hexagonal shape is a double-hexagon formed of two identical convex hexagons sharing two adjacent vertices at their common side. The double-hexagon may be obtainable by mirroring one convex hexagon on one of its sides (mirror axis), wherein the mirror axis then includes the common side. In certain embodiments, the double hexagon is dividable at the common side to obtain two equal convex hexagons, which may be applied either together or separately. Thus, in a particular embodiment, the hexagonal shape is a double-hexagon formed of two identical convex hexagons sharing two adjacent vertices at their common side, wherein said common side is perforated for easy tear-off.

In certain embodiments, the hexagonal shape is a hexagon or a double-hexagon, in particular a convex hexagon or a double-hexagon formed of two identical convex hexagons sharing two adjacent vertices at their common side, wherein preferably the hexagon or double-hexagon has an area of more than 20 cm², such as more than 24 cm², more than 30 cm², or more than 40 cm², and preferably less than 150 cm², such as less than 60 cm², or less than 35 cm².

The hexagonal shapes, in particular the convex hexagons or the double-hexagons, may have mirror symmetry and/or rotational symmetry. Preferred hexagonal shapes are mirror symmetrical with at least one axis of symmetry, such as two, three or four axes of symmetry, in particular six axes of symmetry. Alternatively or additionally preferred hexagonal shapes are rotational symmetrical having an order of at least 2, such as 3 or 4, in particular have 6-fold rotational symmetry. Thus, particularly preferred hexagonal shapes are mirror symmetrical with at least four axes of symmetry and/or have at least 4-fold rotational symmetry, in particular are mirror symmetrical with six axes of symmetry and additionally have 6-fold rotational symmetry.

The hexagon, in particular convex hexagon, according to the invention has three pairs of parallel opposite sites, which may be different or equal in length. In certain embodiments, the two sides of each pair of parallel opposite sides are equal in length, i.e. the hexagon is a parallelogon. The parallelogon may be obtained by elongating a parallelogram, having 2-fold rotational symmetry, or by elongating a rhombus, having 2-fold rotational symmetry and additionally being mirror symmetrical with two axes of symmetry.

In certain embodiments, the six sides of the hexagon, in particular convex hexagon, are equal in length, i.e. the hexagon is equilateral. Alternatively, the hexagon is non-equilateral and has three sides of equal length and three other sides of other equal length. The three sides of equal length and the three other sides of other equal length preferably alternate. Such hexagons are preferably mirror-symmetrical with three axes of symmetry. In another alternative, the hexagon is non-equilateral and has four sides of equal length and two other sides of other equal lengths. This includes in particular a hexagon obtained by elongating a rhombus.

In certain embodiments, the hexagon is non-equilateral and the ratio of shortest side to longest side is 1:4 or less, 1:3 or less, 1:2 or less, 1:1.5 or less, or is about 1:1.

The sides of the hexagon according to the invention have a length of from 0.2 to 10 cm. In certain embodiments, the sides of the hexagon have a length of from 0.3 to 8 cm, from 0.5 to 4 cm, from 0.8 to 3.5 cm, or from 0.9 to 2.0 cm. In further embodiments, the sides of the hexagon have a length of from 2.8 to 8 cm, from 2.8 to 7.5 cm, from 2.8 to 4 cm, from 2.8 to 3.5 cm, or from 2.8 to 3.2 cm. In particular embodiments, two, three, four or six sides of the hexagon have a length of about 0.5 cm, about 0.9 cm, about 1.5 cm, about 1.8 cm, about 2.8 cm, or about 3.2 cm. For example,
two sides of the hexagon have a length of about 0.5 cm, about 0.9 cm or about 1.5 cm, and four sides of the hexagon have a length of about 1.8 cm, about 2.8 cm or about 3.2 cm, or
three sides of the hexagon have a length of about 0.5 cm, about 0.9 cm or about 1.5 cm, and three sides of the hexagon have a length of about 1.8 cm, about 2.8 cm or about 3.2 cm, or
four sides of the hexagon have a length of about 0.5 cm, about 0.9 cm or about 1.5 cm, and two sides of the hexagon have a length of about 1.8 cm, about 2.8 cm or about 3.2 cm.

The height of the hexagon may be in the range of from 0.3 to 17 cm, from 0.8 to 12.5 cm, from 1.3 to 6 cm, or from 1.5 to 3.5 cm. The width of the hexagon may be in the range of from 0.4 to 20 cm, from 1 to 15 cm, from 1.6 to 7 cm, or from 1.8 to 4 cm.

In certain embodiments, the hexagon has an aspect ratio (height-to-width-ratio) of 4:1 or less, 3:1 or less, 2:1 or less, 1.5:1 or less, or 13:2 or less. In particular embodiments, the aspect ratio of the hexagon is 3:2 or less.

In certain embodiments, the hexagon, in particular convex hexagon, is equiangular. Such convex hexagons have each interior angle equal to 120°. Alternatively, the hexagon is non-equiangular, and the smallest angle is 60° or larger, 80° or larger, 90° or larger, or 110° or larger. In particular, the smallest angle is 60° or larger, 80° or larger, 90° or larger, or 110° or larger, and less than 120°. In particular embodiments, the hexagon has two interior angles of equal size (smaller angles) and four other interior angles of other equal size (larger angles), wherein the smaller angles are about 90°.

In a further embodiment, the hexagon, in particular convex hexagon, is regular. Such regular hexagons are preferably mirror symmetrical with six axes of symmetry and additionally have a 6-fold rotational symmetry. In one embodiment, the hexagonal shape is a double-hexagon formed of two identical convex hexagons sharing two adjacent vertices at their common side, wherein the two identical convex hexagons are regular.

Further, according to certain embodiments, the hexagonal shape has an area of more than 20 cm², such as more than 24 cm², more than 30 cm², or more than 40 cm², and preferably less than 150 cm², such as less than 60 cm², or less than 35 cm².

### Medical Patch and Sheet of Medical Patches

In accordance with the invention, the self-adhesive layer structure as described above is for use in a medical patch, which comprises the self-adhesive layer structure arranged on a release liner. The self-adhesive layer structure is also for use in a sheet of medical patches, which comprises a number of self-adhesive layer structures arranged on a release liner.

The medical patch(es) can be either (a) topical medical patch(es) or (a) transdermal therapeutic system(s). In certain embodiments, the medical patch(es) is/are a topical medical patch(es), in particular for the topical administration of capsaicin.

In one embodiment, the invention relates to a medical patch comprising
the self-adhesive layer structure as described above and
a release liner,
wherein the release liner is coextensive with the self-adhesive layer structure or extends beyond the boundary of the self-adhesive layer structure in all directions.

In another embodiment, the invention relates to a sheet of medical patches comprising
two or more self-adhesive layer structured as described above and
a release liner,
wherein the release liner is coextensive with the self-adhesive layer structures or extends beyond the boundary of the self-adhesive layer structure in all directions.

The release liner protects the self-adhesive layer structure(s) and has to be removed before application. The self-adhesive layer structure(s) may be easily peeled-off the release liner to be applied to the skin of a patient—either separately or jointly. In a certain embodiment, the release liner in a sheet of medical patches is not intended to be separated jointly with the self-adhesive layer structure(s), but remains intact after removal of one or more of the self-adhesive layer structure(s) from the sheet. This is of advantage since it is easier to peel off a self-adhesive layer structure from a release liner that extends the area of the layer structure, and because there is no need to dispose of the release liner for each of the self-adhesive layer structures separately. Thus, in such an embodiment, the release liner does not comprise any means for tearing-off parts of the release liner jointly.

Where the release liner extends beyond the boundary of the self-adhesive layer structure, the release liner may be covered by a border layer structure which has the same layer design as the self-adhesive layer structure (i.e., comprising backing layer, active layer comprising a polymer I and an active agent, and optionally a skin contact layer) and identical layer thicknesses and compositions, and which adjoins the self-adhesive layer structure(s) and is coextensive with the outer boundary of the release liner. Such a sheet of medical patches can be conveniently prepared by providing first a sheet of the desired layer structure laminated with the release liner, and then partitioning the layer structure by controlled-depth punching, cutting or slitting through the backing layer, the active layer as well as the optional skin contact layer (if present), but preserving at least part of the release liner, so as to obtain a number of self-adhesive layer structures arranged on a release liner surrounded by the border layer structure.

By using a number of self-adhesive layer structures, the surface of the skin area to be treated may be spanned by puzzling the self-adhesive layer structures side by side without wrinkling. In doing so, the hexagonal shape(s) of the self-adhesive layer structures allows for avoiding gaps and/or overlaps.

The number of self-adhesive layer structures provided to be peeled-off from the release liner depends on the size(s) of the self-adhesive layer structures. Suitable sheets of medical patches comprises from 2 to 400, from 4 to 300, from 6 to 120, or 8 to 30 self-adhesive layer structures. In certain embodiments, the sheet of medical patches comprises 2 to 15 or 150 to 300 self-adhesive layer structures. In particular embodiments, the sheet of medical patches comprises 3, 4, 5, 6, 7 or 8 self-adhesive layer structures. Alternatively, the sheet of medical patches may comprise 150, 180, 200, 240 or 300 self-adhesive layer structures. The self-adhesive layer structures may be equal or different.

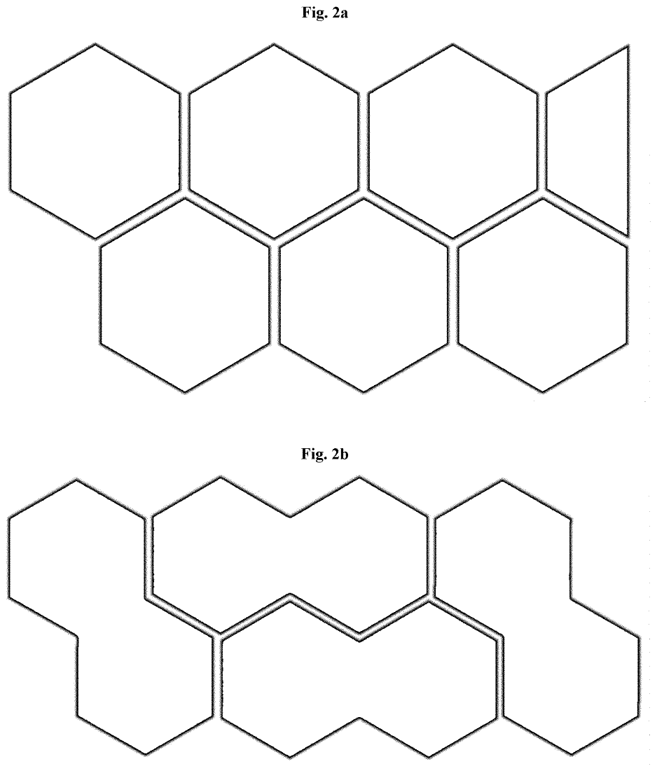

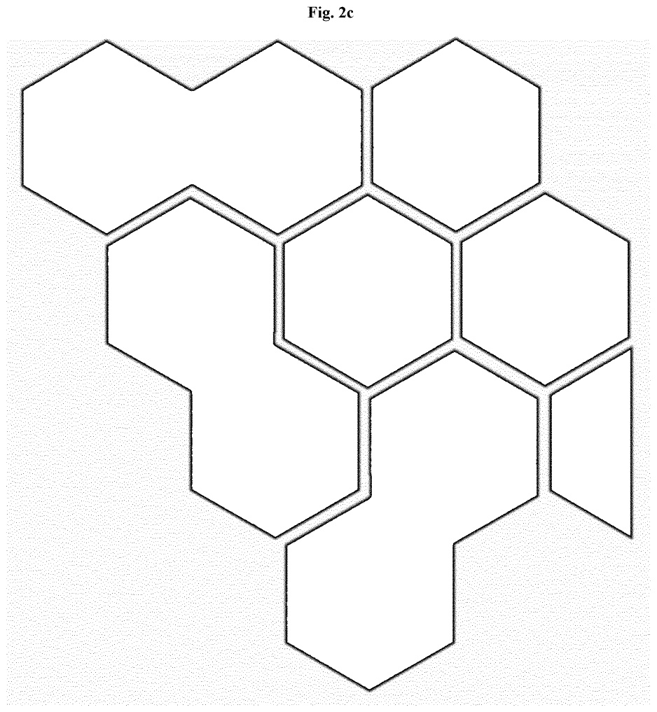

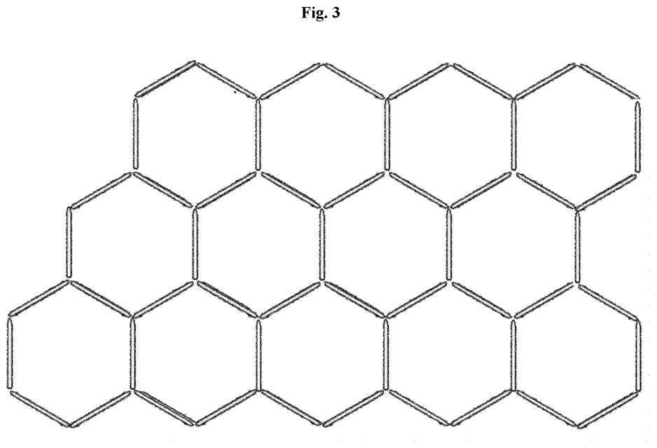

The self-adhesive layer structures may be arranged on the release liner in any pattern, either adjoining each other (tiling the plane) or providing tiny gaps to facilitate grabbing of single self-adhesive layer structures (as shown in FIG. 2a, 2b and 2c). In certain embodiments, the self-adhesive layer structures are arranged on the release liner in a space-saving manner. In particular, the self-adhesive layer structures are arranged side by side on the release liner. Thus, the self-adhesive layer structurers may be arranged in two or more parallel rows with respect to the longitudinal axis of the release liner, wherein preferably each row comprises from 2 to 20, from 3 to 12, or from 4 to 8 self-adhesive layer structures. For example, the self-adhesive layer structures may be arranged in 20 rows, wherein each row comprises 15 self-adhesive layer structures, in particular 15 equal self-adhesive layer structures.

In certain embodiments, the self-adhesive layer structures tile the plane. Preferred self-adhesive layer structures tiling the plane are parallelogons, in particular regular hexagons. The self-adhesive layer structures may also have hexagonal shapes selected from regular hexagons and/or double-hexagons formed of two identical regular hexagons sharing two adjacent vertices and their common side. The self-adhesive layer structures may be separated from each other or connected to each other.

In certain embodiments, the self-adhesive layer structures adjoin each other by sharing two adjacent vertices and their common side and are separated from each other by the common side being cut for independent peel-off from the release liner. Alternatively, the self-adhesive layer structures adjoin each other by sharing two adjacent vertices and their common side and are connected to each other by the common side, which is weakened for easy tear-off. In particular, the common side is perforated for easy tear-off. The sheet of medical patches may also comprise the self-adhesive layer structures adjoining each other by sharing two adjacent vertices and their common sides, wherein some of which are separated from each other by the common side being cut for independent peel-off from the release liner, and some of which are connected to each other by the common side which is weakened for easy tear-off. For example, the self-adhesive layer structures may be arranged in two or more parallel rows with respect to the longitudinal axis of the release liner, wherein each row comprises from 2 to 20 self-adhesive layer structures that adjoin each other by sharing two adjacent vertices and their common side, wherein the rows are separated from each other for independent peel-off and the self-adhesive layer structures within a row are connected to each other by the common side, which is perforated for easy tear-off. In particular embodiments, all self-adhesive layer structures are separated from each other by the common side being cut for independent peel-off from the release liner.

In certain embodiments, the self-adhesive layer structures are connected to each other by at least one and preferably two or more common fastening bridges for joint peel-off from the release liner. The fastening bridge(s) provide(s) (a) single point(s), at which the self-adhesive layer structures are connected to each other, even if they are separated from each other by the common side being cut at least in part. This enables joint peel-off of the thus connected self-adhesive layer structures from the release liner to be applied to the skin of the patient, which is particularly advantageous in case of a large number of and/or small area self-adhesive layer structures. Alternatively, some fastening bridge(s) may be undone, e.g. teared off, for joint peel-off of a lower number of thus connected self-adhesive layer structures. In certain embodiments, the common fastening bride is provided at a vertex and connects at least two and preferably three self-adhesive layer structures. Alternatively, the common fastening bridge may be provided at a side and connect two self-adhesive layer structures. In a preferred sheet of medical patches, neighboring self-adhesive layer structures are all connected to each other in pairs by at least two common fastening bridges provided at two adjacent vertices or at their common side, preferably at the two adjacent vertices. In particular embodiments, neighboring self-adhesive layer structures are all connected to each other in threes by a common fastening bridge provided at the common vertex. This relates in particular to self-adhesive layer structures tiling the plane. Thus, in one embodiment of the sheet of medical patches according to the invention, the self-adhesive layer structures are regular hexagons and are connected to each other by at least one fastening bridge for joint peel-off from the release liner, wherein neighboring self-adhesive layer structures are all connected to each other in threes by a common fastening bridge provided at the common vertex (as shown in FIG. 3).

In certain embodiments, the self-adhesive layer structures have hexagonal shapes selected from two or three different shapes in total. In particular embodiments, the self-adhesive layer structures have hexagonal shapes comprising convex hexagons and double-hexagons formed of two identical convex hexagons sharing two adjacent vertices and their common side, in particular regular hexagons and double-hexagons formed of two identical regular hexagons. Alternatively, the self-adhesive layer structures all have the same hexagonal shape. In particular embodiments, the self-adhesive layer structures are double-hexagons formed of two identical convex hexagons sharing two adjacent vertices and their common side, in particular double-hexagons formed of two identical regular hexagons. In this context, the (regular) convex hexagons or the double-hexagons formed of two identical (regular) convex hexagons, respectively, are congruent.

The self-adhesive layer structures of the sheet of medical patches according to the invention may comprise the same or different active agent(s).

In certain embodiments, the medical patch (a single medical patch) comprises the capsaicin in an amount of about 179 mg, or alternatively in an amount of about 60 mg, about 45 mg, about 30 mg, about 25.5 mg, about 10 mg, or about 1 mg. Likewise, the sheet of medical patches (all medical patches of the sheet as a whole) may comprise the capsaicin in an amount of about 179 mg or less. Preferred sheets of medical patches comprise
3 self-adhesive layer structures, each comprising the capsaicin in an amount of about 60 mg, or
4 self-adhesive layer structures, each comprising the capsaicin in an amount of about 45 mg, or
6 self-adhesive layer structures, each comprising the capsaicin in an amount of about 30 mg, or
7 self-adhesive layer structures, each comprising the capsaicin in an amount of about 25.5 mg.
   Alternatively preferred sheets of medical patches comprise up to 300 self-adhesive layer structures, each comprising the capsaicin in an amount of about 0.6 mg, wherein preferably the self-adhesive layer structures are regular hexagons and are connected to each other by common fastening bridges for joint peel-off from the release liner, wherein neighboring self-adhesive layer structures are all connected to each other in threes by a common fastening bridge provided at the common vertex.

In certain embodiments, the medical patch (a single medical patch) has an area of release of from 0.1 cm² to 280 cm², from 0.6 cm² to 150 cm², or from 1.5 cm² to 35 cm². In particular embodiments, the medical patch has an area of more than 20 cm², such as more than 24 cm², more than 30 cm², or more than 40 cm², and preferably less than 150 cm², such as less than 60 cm², or less than 35 cm². In certain embodiments, the sheet of medical patches (all medical patches of the sheet as a whole) has an area of release of from 1 cm² to 300 cm².

### Method of Treatment/Medical Use

The medical patch or the sheet of medical patches according to the present invention may be suitable for use in a method of treatment, and in particular in a method of treating a human patient. The conditions and diseases to be treated depend on the active agent contained in the patch.

If the active agent is capsaicin, the medical patch or the sheet of medical patches according to the invention is particularly suitable for use in a method of treating neuropathic pain, in particular chronic neuropathic pain, preferably including postherpetic neuralgia, post-surgical neuralgia such as, e.g., post-herniotomy pain, post-thoracotomy pain or post-mastectomy pain, post-traumatic neuropathy, polyneuropathy such as, e.g., painful diabetic neuropathy, chemotherapy-induced neuropathy, neuropathy caused by tumors, HIV-associated neuropathy, alcohol-related neuropathy, small-fiber neuropathy or complex regional pain syndrome, radiculopathy, or compression syndromes such as carpal tunnel syndrome, further preferably peripheral neuropathic pain, neuropathic pain associated with postherpetic neuralgia or diabetic peripheral neuropathy (DPN) of the hands and feet, post-surgical neuropathic pain, joint pain, or cancer pain.

In connection with the above, the medical patch or (parts of) the sheet of medical patches according to the invention is/are preferably applied to at least one body surface on the patient, in particular selected from the back, the bottom, the legs, the feet, or the hands. The preferred application time of a medical patch or sheet of medical patches according to the invention is less than or about 60 minutes on the back, bottom or legs, and less than or about 30 minutes on the feet or hands.

### Process of Manufacture

The medical patch(es) or sheet(s) of medical patches according to the present invention may be manufactured by a conventional manufacturing process such as a solvent-casting process comprising the steps of preparing a coating composition comprising all components of the active layer, and coating and drying the coating composition.

In certain embodiments, in particular in case of capsaicin-containing patches, the process for manufacturing a medical patch or sheet of medical patches according to the present invention comprises the steps of
A. 1.1) coating an active agent-containing coating composition comprising
   (i) a polymer I, and
   (ii) an active agent
   on a release liner;
1.2) drying the coated coating composition to provide an active agent-containing self-adhesive layer structure;
1.3) laminating the active agent-containing self-adhesive layer structure with a backing layer.

The polymer I is in particular at least one silicone-based polymer which is preferably non-curing and therefore typically applied by a solvent-based process. Accordingly, the at least one silicone-based polymer is preferably provided in a solvent, wherein the solids content in the solvent is preferably from 40 to 75% by weight. The solvent is preferably selected from alcoholic solvents, in particular methanol, ethanol, isopropanol and mixtures thereof, and from non-alcoholic solvents, in particular ethyl acetate, hexane, heptane, petroleum ether, toluene, and mixtures thereof, and is more preferably selected from non-alcoholic solvents, and is most preferably ethyl acetate or n-heptane.

The active agent is in particular capsaicin and is preferably homogeneously dissolved or dispersed in the active agent-containing coating composition. In certain embodiments, the capsaicin is provided in an amphiphilic solvent, such as, e.g., diethylene glycol monoethyl ether, 1,3-butanediol, dipropylene glycol or 2, 2-dimethyl-4-hydroxymethyl-1, 3-dioxolane, and the capsaicin preparation is dispersed in the capsaicin-containing coating composition in the form of small droplets (microreservoir system). The amphiphilic solvent must not mix or may only mix to a small extend with the solvent for the silicone-based polymer.

The coated active agent-containing coating composition is solidified by drying. Drying is preferably performed at a temperature of from 20 to 60° C., or from 30 to 40° C.

In case of patches additionally comprising a skin contact layer, these may be manufactured using a process comprising the steps of
A. 1.1) coating an active agent-containing coating composition comprising
   (i) a polymer I, and
   (ii) an active agent
   on a first foil;
1.2) drying the coated coating composition to form the active layer;
1.3) laminating the active layer with a backing layer;
2.1) coating an active-free coating composition comprising
   (i) at least one alkenyl-substituted polydiorganosiloxane,
   (ii) at least one organosiloxane, which contains silicone-bonded hydrogen atoms, and
   (iii) at least one catalyst for the reaction of the SiH groups with the Si-alkenyl groups, on a second foil;
2.2) crosslinking the active-free coating composition at a temperature of from 50° C. to 150° C. or by applying UV light to form the skin contact layer;
2.3) laminating the skin contact layer with a release liner.

The active-free coating composition forms the silicone gel adhesive of the skin contact layer upon curing, i.e. crosslinking of the reactive groups of the silicone polymers. Crosslinking is preferably performed at a temperature of from 40° C. to 140° C.

The active layer and the skin contact layer are preferably prepared separately as indicated above, and then laminated together by removing the foils and then laminating the open sides of the two layers together, so as to give a self-adhesive layer structure of the medial patch, or two or more self-adhesive layer structures of the medical patches constituting the sheet of medical patches. Accordingly, the process may further comprise the steps of
A. 3.1) removing the foils from the active layer and the skin contact layer;
3.2) laminating the open side of the active layer onto the open side of the skin contact layer to provide an active agent-containing self-adhesive layer structure.
   The preparation of the active layer may be performed before or after the preparation of the skin contact layer, or the preparation of the two layers may be performed in parallel.

The active agent-containing self-adhesive layer structure is then preferably partitioned into one or more self-adhesive layer structure(s), having a hexagonal shape, according to the present invention, by, e.g., commonly punching or cutting the backing layer and the active layer, as well as optionally the skin contact layer, and preserving the release liner. Thus, the process for manufacturing a medical patch or sheet of medical patches according to the present invention may further comprise the step of
B. partitioning at least one hexagonal shape out from the active agent-containing self-adhesive layer structure to obtain the medical patch or sheet of medical patches.

In certain embodiments, partitioning is carried out by punching, in particular by punching using steel rule dies. In some embodiments, the punching tool may be discontinuous, in order to provide fastening bridges.

The present invention also relates to a medical patch or sheet of medical patches obtainable by the above described process.

### EXAMPLES

The present invention will now be more fully described with reference to the accompanying examples. It should be understood, however, that the following description is illustrative only and should not be taken in any way as a restriction of the invention. Numerical values provided in the examples regarding the amount of ingredients in the composition or the area weight may vary slightly due to manufacturing variability.

### Example 1

### Preparation of a Sheet of Capsaicin-Containing Medical Patches

The medical patches according to Example 1 may or may not comprise an additional skin contact layer. Thus, the steps of preparing and coating an active-free coating composition and laminating the resulting active-free layer with the previously prepared capsaicin-containing layer are optionally.

### Capsaicin-Containing Coating Composition

The formulation of the capsaicin-containing coating composition is summarized in Table 1.1 below. The solids %-values refer to the amounts (Amt) in % by weight.

TABLE 1.1

| Ingredient (Trade Name) | Solids [%] |
|---|---|
| Capsaicin | 8 |
| Polysiloxanes in n-heptane. Solids content of | 70 |
| 70% by weight (Liveo ™ BIO-PSA 7-4301 | |
| and BIO-PSA 7-4201) | |
| Transcutol (diethylene glycol monoethyl ether) | 19.2 |
| Ethyl cellulose | 0.8 |
| Silicone oil 360MED Fluid 12.500 cSt. | 2 |
| Total | 100 |
| Area Weight [g/m²] | 80 |
| Capsaicin content [μg/cm²] | 640 |

### Preparation of the Capsaicin-Containing Coating Composition

Transcutol was initially thickened with the ethyl cellulose under stirring (100-300 rpm).

A vessel was loaded with the polysiloxane mixture and the silicone oil and stirred (100-300 rpm) at least for 5 min before the ethyl cellulose/Transcutol solution was added. After further 10 min of stirring (100-300 rpm) capsaicin was added. The mixture was then stirred at approx. 250-300 rpm until a homogeneous mixture was obtained (at least 60 min).

### Coating of the Capsaicin-Containing Coating Composition

The resulting capsaicin-containing coating composition was coated on a fluoropolymer coated polyester film (Scotchpak™ 1022). The solvent was removed at room temperature for about 20-30 min.

The coating thickness was chosen such that removal of the solvent results in an area weight of the capsaicin-containing layer of about 80 g/m².

The resulting capsaicin-containing microreservoir layer was then laminated with a backing layer (polyester film, 19 μm).

Optionally, the adhesively equipped foil used for the coating and drying of the capsaicin-containing layer was removed to obtain a capsaicin containing self-adhesive layer structure comprising the backing layer and the capsaicin-containing layer, wherein the capsaicin-containing layer is attached to the backing layer.

### Active-Free Coating Composition

For Example 1, the formulation of the active-free coating composition is summarized in Table 1.2 below. The solids %-values refer to the amounts (Amt) in % by weight.

TABLE 1.2

| | Amt | Solids |
|---|---|---|
| Ingredient (Trade Name) | [g] | [%] |
| Component A of Soft Skin Adhesive Kit | 25 | 50 |
| (Liveo ™ MG 7-9900 (A)) | | |
| Component B of Soft Skin Adhesive Kit | 25 | 50 |
| (Liveo ™ MG 7-9900 (B)) | | |
| Total | 50 | 100 |

| | |
|---|---|
| Area Weight [g/m²] | 230 |

### Preparation of the Active-Free Coating Composition

Both components were weighed separately, and then component A was added to the mixing vessel followed by component B. Then the mixture was mixed at approx. 200 rpm for approx. 5 min until a homogeneous mixture of Component A and Component B was obtained.

### Coating of the Active-Free Coating Composition

Within a time frame of approx. 30 min, the resulting active-free coating composition was coated on an abhesively equipped foil. The coating temperature was set to 120° C. The resulting active-free layer was heated at this temperature for approx. 40 min.

The coating thickness was chosen such that removal of the solvents resulted in a layer thickness of the active-free (skin contact) layer of approx. 230 g/m².

The resulting active-free (skin contact) layer was laminated with a release liner (FEP, fluorinated ethylene propylene, 125 μm).

### Lamination of the Capsaicin-Containing Layer and the Active-Free (Skin Contact) Layer

The active-free (skin contact) layer was then laminated with the capsaicin-containing layer. For this purpose, the adhesively equipped foils used for the coating and drying of the layers were removed and the resulting open sides of the active-containing layer and the active-free (skin contact) layer were laminated together resulting in a capsaicin-containing self-adhesive layer structure comprising the backing layer, the capsaicin-containing layer, and the active-free (skin contact) layer, wherein the capsaicin-containing layer is attached to the backing layer, and the active-free (skin contact) layer is attached to the capsaicin-containing layer, and wherein the structure is closed by a release liner, which is attached to the active-free (skin contact) layer.

### Preparation of the Medical Patches and the Sheet of Medical Patches

The individual medical patches were punched out from the capsaicin-containing self-adhesive layer structure obtained as described, either comprising the backing layer and the capsaicin-containing layer, or the backing layer, the capsaicin-containing layer and the active-free (skin contact) layer, without impairing the common release liner.

Then, the sheet of medical patches was sealed into pouches of the primary packaging material.

### Examples 2A-M and Reference Example

### Evaluating the Performance of Placebo Medical Patches and Sheets of Placebo Medical Patches

Different medical patches and sheets of medical patches according to the invention (but not containing an active agent) were prepared based on an active-free adhesive layer (Examples 2A-M). A medical patch having a rectangular shape of 20 cm×14 cm was prepared in the same manner (Reference Example).

### Coating Composition

The formulation of the coating composition is summarized in Table 2.1 below. The solids %-values refer to the amounts (Amt) in % by weight.

TABLE 2.1

| | Amt | Solids |
|---|---|---|
| Ingredient (Trade Name) | [g] | [%] |
| Polysiloxanes in n-heptane. Solids content of | 1.73 | 77.2 |
| 70% by weight (Liveo ™ BIO-PSA 7-4301 | | |
| and BIO-PSA 7-4201) | | |
| Transcutol (diethylene glycol monoethyl ether) | 0.45 | 20.0 |
| Ethyl cellulose | 0.02 | 0.8 |
| Silicone oil 360MED Fluid 12.500 cSt. | 0.04 | 2.0 |
| Total | 2.24 | 100 |

| | |
|---|---|
| Area Weight [g/m²] | 80 |

### Preparation of the Medical Patches and the Sheet of Medical Patches

The individual medical patches were punched out from the active-free self-adhesive layer structure obtained as described above located on a release liner, comprising the backing layer and the active-free layer, without impairing the common release liner. Medical patches and sheets of medical patches having a hexagonal shape as indicated in table 2.2 were obtained in this way.

Then, the performance of the medical patches and sheets of medical patches was evaluated by applying the respective medical patch(es) or sheet of medical patches to different skin areas, in particular to an ankle/a finger and evaluating the coverage of the skin area to be treated (+: poor coverage to +++: good coverage) as well as the occurrence of wrinkling (*: no/low wrinkling to ***: a lot of wrinkling).

The results are shown in Table 2.2 below.

TABLE 2.2

| Medical patches/ | | Coverage | | Wrinkling | |
|---|---|---|---|---|---|
| sheets of medical patches | | Ankle | Finger | Ankle | Finger |
| Ex. 2A | Regular hexagon | + | too big | *** | too big |
| | (side length = 7.5 cm) | | | | |
| Ex. 2B | Regular hexagon | ++ | ++ | ** | ** |
| | (side length = 3.2 cm) | | | | |
| Ex. 2C | Double-hexagon, not separated | ++ | too big | *** | too big |
| | (side length = 3.2 cm) | | | | |
| Ex. 2D | Regular hexagon | ++ | ++ | ** | ** |
| | (side length = 2.8 cm) | | | | |
| Ex. 2E | Double-hexagon, not separated | ++ | too big | *** | too big |
| | (side length = 2.8 cm) | | | | |
| Ex. 2F | 2 regular hexagons in 2 rows | +++ | +++ | * | * |
| | (side length = 1.8 cm) | | | | |
| Ex. 2G | 2 regular double-hexagons in 2 rows | +++ | +++ | * | * |
| | (side length = 1.8 cm) | | | | |
| Ex. 2H | 3 regular hexagons in 2 rows | +++ | +++ | * | * |
| | (side length = 1.55 cm) | | | | |
| Ex. 2I | 2 double-hexagons in 2 rows | +++ | +++ | * | * |
| | (side length = 1.55 cm) | | | | |
| Ex. 2J | 9 regular hexagons in 3 rows, | ++ | too big | ** | too big |
| | connected by fastening bridges | | | | |
| | (side length = 0.9 cm) | | | | |
| Ex. 2K | 9 regular hexagons in 3 rows | +++ | too big | * | too big |
| | (side length = 0.9 cm) | | | | |
| Ex. 2L | 9 regular hexagons in 3 rows, | +++ | +++ | * | * |
| | combination of single and double | | | | |
| | hexagons | | | | |
| | (side length = 0.9 cm) | | | | |
| Ex. 2M | 4 regular hexagons in 3 rows | +++ | +++ | * | * |
| Ref. Ex. | Rectangle | too big | too big | too big | too big |
| | (20 cm × 14 cm) | | | | |

In general, medical patches/sheets of medical patches having a hexagonal shape showed improved coverage and lower wrinkling than the medical patch having a rectangular shape. The different medical patches/sheets of medical patches having a hexagonal shape performed similarly good on substantially plane or cylindrical surfaces, such as back, thighs, lower legs or arms.

On uneven or curved surfaces, such as on an ankle or finger, single hexagons showed a slightly better performance than double hexagons. Smaller hexagonal shapes provided better coverage and lower wrinkling compared to larger hexagonal shapes. The best results in handling were obtained with hexagonal shapes having a side length of 1.55 cm or 1.8 cm. Hexagonal shapes having a side length of 0.9 cm were inferior in handling but provided very good coverage and low wrinkling.

In case of curved surfaces, gapless coverage could only be achieved by minimally overlapping the edges of the hexagonal shapes.

In addition, it has been shown that hexagonal shapes connected by fastening bridges were very good in handling. Single hexagonal shapes or groups of hexagonal shapes could be separated (without a cutting tool), or all the hexagonal shapes of the sheet of medical patches could be applied together.

Selection of the most useful medical patch(es)/sheet of medical patches depends on the respective skin area to be treated.

The invention relates in particular to the following further items:

1. A self-adhesive layer structure for use in a medical patch, having a hexagonal shape and comprising:
A) a backing layer; and
B) an active layer comprising a polymer I and an active agent;
wherein
   the backing layer and the active layer are coextensive and provide the hexagonal shape of the self-adhesive layer structure,
   the hexagonal shape comprises at least one hexagon, wherein
      all pairs of opposite sides of the hexagon are parallel, and
      the sides of the hexagon have a length of from 0.2 to 10 cm.

2. Self-adhesive layer structure according to item 1, wherein
the self-adhesive layer structure is a pressure sensitive adhesive layer structure.

3. Self-adhesive layer structure according to item 1 or 2, wherein
the at least one hexagon is at least one convex hexagon.

4. Self-adhesive layer structure according to item 3, wherein
the hexagonal shape comprises one or two convex hexagons.

5. Self-adhesive layer structure according to any of items 1 to 4, wherein
the hexagonal shape is a convex hexagon.

6. Self-adhesive layer structure according to any of items 1 to 4, wherein
the hexagonal shape is a double-hexagon formed of two identical convex hexagons sharing two adjacent vertices and their common side.

7. Self-adhesive layer structure according to item 6, wherein
said common side is perforated for easy tear-off.

8. Self-adhesive layer structure according to any of items 1 to 7, wherein
the hexagon is a parallelogon.

9. Self-adhesive layer structure according to any of items 1 to 8, wherein
the hexagon is equilateral, or
the hexagon is non-equilateral and has
   three sides of equal length and three other sides of other equal length, or
   four sides of equal length and two other sides of other equal length.

10. Self-adhesive layer structure according to any of items 1 to 9, wherein
the hexagon is non-equilateral and the ratio of shortest side to longest side is 1:4 or less, 1:3 or less, 1:2 or less, 1:1.5 or less, or is about 1:1.

11. Self-adhesive layer structure according to any of items 1 to 10, wherein
the sides of the hexagon have a length of from 0.3 to 8 cm, from 0.5 to 4 cm, from 0.8 to 3.5 cm, or from 0.9 to 2 cm.

12. Self-adhesive layer structure according to any of items 1 to 11, wherein
two, three, four or six sides of the hexagon have a length of about 0.5 cm, about 0.9 cm, about 1.5 cm, about 1.8 cm, about 2.8 cm, or about 3.2 cm.

13. Self-adhesive layer structure according to any of items 1 to 12, wherein
the hexagon has an aspect ratio of 4:1 or less, 3:1 or less, 2:1 or less, 1.5:1 or less, or √3:2 or less.

14. Self-adhesive layer structure according to any of items 1 to 13, wherein
the hexagon is equiangular.

15. Self-adhesive layer structure according to any of items 1 to 13, wherein
the hexagon is non-equiangular, and
the smallest angle is 600 or larger, 800 or larger, 900 or larger, or 1100 or larger.

16. Self-adhesive layer structure according to any of items 1 to 9 and 11 to 14, wherein
the hexagon is regular.

17. Self-adhesive layer structure according to any of items 1 to 15, wherein
the active agent is at least one analgesic.

18. Self-adhesive layer structure according to item 17, wherein
the active agent is selected from the group consisting of buprenorphine, capsaicin, diclophenac, fentanyl, ibuprofen, and lidocaine.

19. Self-adhesive layer structure according to item 17 or 18, wherein
the active agent is capsaicin.

20. Self-adhesive layer structure according to item 19, wherein
the active layer comprises the capsaicin in a concentration of from 2 to 20 wt-%, from 5 to 15 wt-%, from 5 to 10 wt-%, or about 8 wt-%.

21. Self-adhesive layer structure according to item 19 or 20, wherein
the self-adhesive layer structure comprises the capsaicin in an amount of from 0.5 to 180 mg, from 1.2 to 90 mg, or 19 to 45 mg.

22. Self-adhesive layer structure according to any of items 19 to 21, wherein
the self-adhesive layer structure comprises the capsaicin in an amount of about 179 mg, about 60 mg, about 45 mg, about 30 mg, about 25.5 mg, about 10 mg, or about 1 mg.

23. Self-adhesive layer structure according to any of items 1 to 22 for transdermal or topical delivery of the active agent.

24. Self-adhesive layer structure according to any of items 1 to 23, wherein
the polymer I is a pressure sensitive adhesive polymer.

25. Self-adhesive layer structure according to any of items 1 to 24, wherein
the polymer I is selected from silicone-based polymers, acrylic polymers, silicone acrylic hybrid polymers, and polymers based on natural or synthetic rubbers.

26. Self-adhesive layer structure according to any of items 1 to 25, wherein
the polymer I is a silicone-based polymer obtainable by polycondensation of silanol endblocked polydimethylsiloxane with a silicate resin.

27. Self-adhesive layer structure according to any of items 1 to 26, comprising
A) the backing layer;
B) the active layer; and
C) a skin contact layer;
wherein
   the backing layer, the active layer and the skin contact layer are coextensive and provide the hexagonal shape of the self-adhesive layer structure.

28. Self-adhesive layer structure according to item 27, wherein
the skin contact layer is adhesive and preferably is pressure sensitive adhesive.

29. Self-adhesive layer structure according to item 27 or 28, wherein
the skin contact layer comprises a polymer II.

30. Self-adhesive layer structure according to item 29, wherein
the polymer II is a polymer or a mixture of polymers in which the active agent is substantially insoluble.

31. Self-adhesive layer structure according to item 29 or 30, wherein
the polymer II is a pressure sensitive adhesive or a mixture of pressure sensitive adhesives.

32. Self-adhesive layer structure according to any of items 29 to 31, wherein
the polymer II is a polymer or a mixture of polymers selected from the group consisting of silicone acrylic hybrid polymers, silicone-based polymers, silicone gel adhesives, and polymers based on natural or synthetic rubbers.

33. Self-adhesive layer structure according to any of items 29 to 32, wherein
the polymer II is a polymer or a mixture of polymers selected from the group consisting of silicone-based polymers and silicone gel adhesives.

34. Self-adhesive layer structure according to any of items 29 to 33, wherein
the polymer II is a silicone gel adhesive.

35. Self-adhesive layer structure according to item 27 or 28, wherein
the skin contact layer comprises a silicone gel adhesive.

36. Self-adhesive layer structure according to item 34 or 35, wherein
the silicone gel adhesive is obtainable by reacting a gel producing composition comprising (i) at least one alkenyl-substituted polydiorganosiloxane, (ii) at least one organosiloxane, which contains silicone-bonded hydrogen atoms, and (iii) at least one catalyst for the reaction of the SiH groups with the Si-alkenyl groups.

37. Self-adhesive layer structure according to any of item 34 to 36, wherein
the silicone gel adhesive is obtainable by reacting a gel producing composition comprising (i) a copolymer of vinylmethylsiloxane and dimethylsiloxane with (ii) methylhydrogen polysiloxane with trimethylsilyl endgroups in the presence of (iii) a platinum catalyst.

38. Self-adhesive layer structure according to any of items 34 to 37, wherein
the silicone gel adhesive is a silicate resin-reinforced silicone gel adhesive that contains from about 2 to about 45% by weight or from about 20 to about 30% by weight of at least one hydroxyl substituted silicate resin.

39. Self-adhesive layer structure according to any of items 1 to 38, wherein
the saturation concentration of the active agent in the skin contact layer is less than 0.1% by weight, less than 0.05% by weight, less than 0.02% by weight, or less than 0.01% by weight.

40. Self-adhesive layer structure according to any of items 1 to 39, wherein
the active layer comprises further excipients or additives selected from the group consisting of additional polymers, cross-linking agents, crystallization inhibitors, solubilizers, fillers, tackifiers, plasticizers, stabilizers, softeners, substances for skincare, permeation enhancers, pH regulators, and preservatives.

41. Self-adhesive layer structure according to items 40, wherein
the active layer comprises an additional polymer selected from dimethylpolysiloxanes and ethyl celluloses.

42. Self-adhesive layer structure according to item 40 or 41, wherein
the active layer comprises a permeation enhancer selected from diethylene glycol monoethyl ether.

43. A medical patch comprising
the self-adhesive layer structure according to any of items 1 to 42 and
a release liner,
wherein the release liner is coextensive with the self-adhesive layer structure or extends beyond the boundary of the self-adhesive layer structure in all directions.

44. A sheet of medical patches comprising
two or more self-adhesive layer structures according to any of items 1 to 42 and
a release liner,
wherein the release liner is coextensive with the self-adhesive layer structures or extends beyond the boundary formed by all self-adhesive layer structures in all directions.

45. Sheet of medical patches according to item 44, comprising
from 2 to 400, from 4 to 300, from 6 to 120, or 8 to 30 self-adhesive layer structures.

46. Sheet of medical patches according to item 45, comprising
2 to 15 or 150 to 300 self-adhesive layer structures.

47. Sheet of medical patches according to item 45 or 46, comprising
3, 4, 5, 6, 7 or 8 self-adhesive layer structures.

48. Sheet of medical patches according to any of items 44 to 47, wherein
the self-adhesive layer structures are arranged in two or more parallel rows with respect to the longitudinal axis of the release liner, wherein
each row preferably comprises from 2 to 20, from 3 to 12, or from 4 to 8 self-adhesive layer structures.

49. Sheet of medical patches according to any of items 44 to 48, wherein
the self-adhesive layer structures tile the plane.

50. Sheet of medical patches according to any of items 44 to 49, wherein
the self-adhesive layer structures adjoin each other by sharing two adjacent vertices and their common side and are
   separated from each other by the common side being cut for independent peel-off from the release liner, or
   connected to each other by the common side, which is weakened for easy tear-off.

51. Sheet of medical patches according to item 50, wherein
all self-adhesive layer structures are separated from each other by the common side being cut for independent peel-off from the release liner.

52. Sheet of medical patches according to item 51, wherein
the common side is perforated for easy tear-off.

53. Sheet of medical patches according to any of items 44 to 49, wherein
the self-adhesive layer structures are connected to each other by at least one and preferably two or more common fastening bridges for joint peel-off from the release liner.

54. Sheet of medical patches according to item 53, wherein
the common fastening bridge is provided
   at a vertex and connects at least two and preferably three self-adhesive layer structures, or
   at a side and connects two self-adhesive layer structures.

55. Sheet of medical patches according to item 53 or 54, wherein
neighboring self-adhesive layer structures are all connected to each other in pairs by at least two common fastening bridges provided at two adjacent vertices or at their common side, preferably at the two adjacent vertices.

56. Sheet of medical patches according to items 53 to 55, wherein
neighboring self-adhesive layer structures are all connected to each other in threes by a common fastening bridge provided at the common vertex.

57. Sheet of medical patches according to any of items 44 to 56, wherein
the self-adhesive layer structures have hexagonal shapes selected from two or three different shapes in total.

58. Sheet of medical patches according to item 47, wherein
the self-adhesive layer structures have hexagonal shapes comprising convex hexagons and double-hexagons formed of two identical convex hexagons sharing two adjacent vertices and their common side.

59. Sheet of medical patches according to any of items 44 to 56, wherein
the self-adhesive layer structures all have the same hexagonal shape.

60. Sheet of medical patches according to item 59, wherein
the self-adhesive layer structures are double-hexagons formed of two identical convex hexagons sharing two adjacent vertices and their common side.

61. Process for manufacturing a medical patch according to claim 43 or a sheet of medical patches according to any of claims 44 to 60, comprising the steps of:
A. 1.1) coating an active agent-containing coating composition comprising
   (i) a polymer I, and
   (ii) an active agent
   on a release liner;
1.2) drying the coated coating composition to provide an active agent-containing self-adhesive layer structure;
1.3) laminating the active agent-containing self-adhesive layer structure with a backing layer; and
B. partitioning at least one hexagonal shape out from the active agent-containing self-adhesive layer structure to obtain the medical patch or sheet of medical patches.

1. A self-adhesive layer structure for use in a medical patch, having a hexagonal shape and comprising:
A) a backing layer; and
B) an active layer comprising a polymer I and an active agent;
wherein
the backing layer and the active layer are coextensive and provide the hexagonal shape of the self-adhesive layer structure,
the hexagonal shape comprises at least one hexagon, wherein
all pairs of opposite sides of the hexagon are parallel, and
the sides of the hexagon have a length of from 0.2 to 10 cm.

2. Self-adhesive layer structure according to claim 1, wherein
the self-adhesive layer structure is a pressure sensitive adhesive layer structure.

3. Self-adhesive layer structure according to claim 1 or 2, wherein
the at least one hexagon is at least one convex hexagon.

4. Self-adhesive layer structure according to claim 3, wherein
the hexagonal shape comprises one or two convex hexagons.

5. Self-adhesive layer structure according to any of claims 1 to 4, wherein
the hexagonal shape is a convex hexagon.

6. Self-adhesive layer structure according to any of claims 1 to 4, wherein
the hexagonal shape is a double-hexagon formed of two identical convex hexagons sharing two adjacent vertices and their common side.

7. Self-adhesive layer structure according to any of claims 3 to 6, wherein
the hexagon is equilateral, or
the hexagon is non-equilateral and has
three sides of equal length and three other sides of other equal length, or
four sides of equal length and two other sides of other equal length.

8. Self-adhesive layer structure according to any of claims 1 to 7, wherein
the sides of the hexagon have a length of from 0.3 to 8 cm, from 0.5 to 4 cm, from 0.8 to 3.5 cm, or from 0.9 to 2 cm.

9. Self-adhesive layer structure according to any of claims 1 to 8, wherein
the hexagon is regular.

10. Self-adhesive layer structure according to any one of claims 1 to 9, wherein
the active agent is capsaicin, and wherein
the active layer preferably comprises the capsaicin in a concentration of from 1 to 15 wt-%, from 2 to 12 wt-%, from 4 to 10 wt-%, or about 8 wt-%.

11. A medical patch comprising
the self-adhesive layer structure according to any of claims 1 to 10 and
a release liner,
wherein the release liner is coextensive with the self-adhesive layer structure or extends beyond the boundary of the self-adhesive layer structure in all directions.

12. A sheet of medical patches comprising
two or more self-adhesive layer structures according to any of claims 1 to 10 and
a release liner,
wherein the release liner is coextensive with the self-adhesive layer structures or extends beyond the boundary formed by all self-adhesive layer structures in all directions.

13. Sheet of medical patches according to claim 12, comprising from 2 to 400, from 4 to 300, from 6 to 120, or 8 to 30 self-adhesive layer structures, wherein preferably the self-adhesive layer structures tile the plane.

14. Sheet of medical patches according to claim 12 or 13, wherein
the self-adhesive layer structures adjoin each other by sharing two adjacent vertices and their common side and are
separated from each other by the common side being cut for independent peel-off from the release liner, or
connected to each other by the common side, which is weakened for easy tear-off.

15. Sheet of medical patches according to any of claims 12 to 14, wherein
the self-adhesive layer structures are connected to each other by at least one and preferably two or more common fastening bridges for joint peel-off from the release liner, and wherein
the common fastening bridge is preferably provided
at a vertex and connects at least two and preferably three self-adhesive layer structures, or
at a side and connects two self-adhesive layer structures.

16. Sheet of medical patches according to any of claims 12 to 15, wherein the self-adhesive layer structures have hexagonal shapes selected from two or three different shapes in total, or wherein the self-adhesive layer structures all have the same hexagonal shape.

17. Process for manufacturing a medical patch according to claim 11 or a sheet of medical patches according to any of claims 12 to 16, comprising the steps of:
A. 1.1) coating an active agent-containing coating composition comprising
(i) a polymer I, and
(ii) an active agent
on a release liner;
1.2) drying the coated coating composition to provide an active agent-containing self-adhesive layer structure;
1.3) laminating the active agent-containing self-adhesive layer structure with a backing layer; and
B. partitioning at least one hexagonal shape out from the active agent-containing self-adhesive layer structure to obtain the medical patch or sheet of medical patches.
